SEQUENCE LISTING (84 sequences)

SEQ ID NO: 1 - protein, 85 aa, Artificial Sequence
  Consensus Sequence
XXXXXXXXXXXXXXXXXXXXXXXXXDXXGXXXGXXXXXXXXXXXXXXXXXXXXXXXXXXXXXXGXXXXXXXXXXXXXXXXXXXXX

SEQ ID NO: 2 - protein, 73 aa, Fibrobacter succinogenes
GIKNIRFSMTEAESSYSVFDMQGIKLSSFTAKGMNEAMNLVRENAKLRKQAKGVFFVRKNGEKSLTKKVVIHE

SEQ ID NO: 3 - protein, 73 aa, Fibrobacter succinogenes
AIAKVRFDMTEAESNFSVYSMQGQKLGTFTAKGMADAMNLVKTDAKLRKQAKGVFFVRKEGAKLMSKKVVVFE

SEQ ID NO: 4 - protein, 71 aa, Fibrobacter succinogenes
GIKGFRVLGADAVANFDVFDLTGKKVSSFTARNIHEAKKLWRENPQSRNVQGVCIIRNRYNGAVARVRTTR

SEQ ID NO: 5 - protein, 70 aa, Fibrobacter succinogenes
AIRQKIRMNAIAAATYDVFDLTGKKVSSFTAHSMDEAKNMLRGGAQANMQGVCIIRNRGTGMMAKVRTTR

SEQ ID NO: 6 - protein, 70 aa, Fibrobacter succinogenes
AIQSNLRMNYPVLSDYDVFDMNGVRLGRMSAYSVDEAVTTLKNTSAIKVQGIYLLRSVKNGAVKTVRIAR

SEQ ID NO: 7 - protein, 70 aa, Fibrobacter succinogenes
SIQTQVRYEYPEVSDYYVFDVNGVRIGRMSAYTMDEAVATLKNTSAIKVQGLYMLRSVKTGEVKSVRVAR

SEQ ID NO: 8 - protein, 70 aa, Fibrobacter succinogenes
GIKTSVQYQAPRIGSYDVFDANGVRLGRMNAYSMSEAAQILKSSNDVKNNGIYMLRSVQSGAVKSVRISR

SEQ ID NO: 9 - protein, 70 aa, Fibrobacter succinogenes
KFGNKIRLEQNTLQNYDVFDVQGVRLGNLSAYGFSDATAKLKSASTAKTSGIYFLRSRTTGKMQSVRVAR

SEQ ID NO: 10 - protein, 68 aa, Fibrobacter succinogenes
ALPGKIRNLAVAEKNYNVFDAQGKKVGAFTTRGVEDLHAITAGLVKNSGVYIVKAKNGGQAFRITVKK

SEQ ID NO: 11 - protein, 68 aa, Fibrobacter succinogenes
GLRGANFRLPTEAENYSVFDVNGVLVGKFLATTKADVQRMTKSVVRQNGIYFVKSLKSGNAYRISVAK

SEQ ID NO: 12 - protein, 72 aa, Fibrobacter succinogenes
EIEDRSLAIRPKFVASSGPQTYDVFDMLGAHVGRVDAATVQDVAQKVRALVKQNGTYLVKSRNSSATIRVMK

SEQ ID NO: 13 - protein, 71 aa, Fibrobacter succinogenes
PIVGLAKGVRYNVQGVQTYGVYGLNGKFIGRVDASNNFDVRSKVNSLVKESGVYIVKSLTTGNTHRLSVTK

SEQ ID NO: 14 - protein, 72 aa, Fibrobacter succinogenes
SSSSGTDAIPFMAQLFDKAGVYQVFDMQGNFLGKVELKNGLSLKQAVASKFARSSVYLVKRGAFAKTIAVER

SEQ ID NO: 15 - protein, 71 aa, Fibrobacter succinogenes
ESSSSTVALHAAPKMELKSGNFQVFDMQGRFLGTVKLDAGASVAQVLKANFKNAGIYMVKQGNFMQRVAVK

SEQ ID NO: 16 - protein, 69 aa, Fibrobacter succinogenes
TSEASSTALPQFAPKTNTITSAQVFDMQGKFLGNVNATSDIQSAIKNKFHSTGIYMVKLGSAMKSVSVK

SEQ ID NO: 17 - protein, 66 aa, Fibrobacter succinogenes
GLNKVRLTSFESENSYNVFSATGKHLGRVDLNGASMPQALKNAGYARGTYMVRSVKGNQIQRVNVR

SEQ ID NO: 18 - protein, 66 aa, Fibrobacter succinogenes
ALPKSRVELQIPEKIYAVFGMTGKFLGNVEVNGQSVAKSIRAAGFTPGVYMVRSVGQSKTFRVLVK

SEQ ID NO: 19 - protein, 67 aa, Fibrobacter succinogenes
SLAPSLRLKTTAGVYNVYSLTGKRLGFVEIIENDVPNMQKTMKNAGFGKGVYILRNKTRSFMLPVDR

SEQ ID NO: 20 - protein, 67 aa, Fibrobacter succinogenes
GLKPAVKFQANASRVYRVYSVSGKLLGTVELVGKKAAEALQSAGFNKGVYMLKSVDGHKTFMTSVAR

SEQ ID NO: 21 - protein, 70 aa, Fibrobacter succinogenes
ITTSITMKLSRETMVLGNASIFDLQGRYLGNLQAEQLRDGDIAKAIRAKFGKPGVYLVKQEKKMIRILTK

SEQ ID NO: 22 - protein, 76 aa, Fibrobacter succinogenes
GIVSRVAALQLSGVNENFDVFDLNGKHLGFAKVTPSEWNALGHKSLQKTLSASGFNAGMYIVRAKRSHRLVRVNVR

SEQ ID NO: 23 - protein, 72 aa, Fibrobacter succinogenes
SIKPKIAAGAFAKIAGEYKVFDLMGNMLGKVRLNAGASLIEIKNGLKTAGFGRGVYVVRNPAGKPLKLQVGE

SEQ ID NO: 24 - protein, 72 aa, Fibrobacter succinogenes
NIARKIAFEHGPAMVRYQVFDLNGQLIKSANVMAGSVSEAWNLVKTGLRKGAYVMRYSEAGQGSHVVKVRLQ

SEQ ID NO: 25 - protein, 70 aa, Fibrobacter succinogenes
SIRPKTAAVVEPSVYRVDIFDTKGALVRRMNVESAKVSDVAWMTRGLPAGLYVMQLKNANTERRKFITVK

SEQ ID NO: 26 - protein, 297 aa, Fibrobacter succinogenes
MSVERNLKKFMALAGVAAGLSMFAVGANAAPNPNFHIYIAYGQSNMAGNGDIVPSEDQAEAPKNFIMLASHNANASQRSGKTNQSIKTGEWYPAIPPMFHPFENLSPADYFGRAMADSLPGVTVGIIPVAIGAVSIRAFDKDQYEAYFRGDGKDIMNWGWPKDYDNNPPGRILELAKKAKEVGVIKGFIFHQGESDGTDANWRKTVYKTYKDVIDALGLDENEVPFVAGELLQEGQNCCSSKNGGIAQLKQNFKKFGLASSKGLQGNGKDPYHFGRAGVIELGKRYCSEMLKLIDKT

SEQ ID NO: 27 - protein, 285 aa, Fibrobacter succinogenes
MSVEMSFKKLMGIAGVAAGLSMFAVMGANAAPDPNFHIYIAYGQSNMEGNARNFTDVDKKEHPRVKMFATTSCPSLGRPTVGEMYPAVPPMFKCGEGLSVADWFGRHMADSLPNVTIGIIPVAQGGTSIRLFDPDDYKNYLNSAESWLKNGAKAYGDDGNAMGRIIEVAKKAQEKGVIKGIIFHQGETDGGMSNWEQIVKKTYEYMLKQLGLNAEETPFVAGEMVDGGSCAGFSSRVRGLSKYIANFGVASSKGYGSKGDGLHFTVEGYRGMGLRYAQQMLKLIN

SEQ ID NO: 28 - protein, 276 aa, Neocallimastix patriciarum
MRTFAIAAFVATTLSAVSQTFAAPDPNFHIYLAFGQSNMEGQGPIGSQDRTVDKRFQMISTVSGCNGRQMGNWYDAVPPLANCDGKLGPVDYFGRTLVKKLPQEIKVGVAVVAVAGCDIQLFEKNNYRNYRLESYMQGRVNAYGGNPYGRLIEVAKKAQQVGVIKGILLHQGETNTGQQNWPNRVKAVYEDMLKDLGLNAKDVPLLAGEVVQSNQGGQCGSMNSIIQKLPSVIPTAHVISSQGLGQQGDGLHFSSQAYRTFGERYADEMLKILGDV

SEQ ID NO: 29 - protein, 273 aa, Orpinomyces sp.
MRTSVVITFLAAALTVMAKPHAKPDPNFHIYLALGQSNMEGQGNVEAQDRVEDKRFKLISTADECMGRELGEWYPALPPIVNCYGNLGPVDYFGRTLTKKLPKEVKVGVCAVAVAGCDIQLFEEENYKSYEIPDWMQGRIDHYGGNPFRRLVNIAKKAQKAGVIKGILLHQGETNNGQEDWPKRIKVVYERLLKELNLKAEEVPLLAGEVVREEYEGMCSLHNTVIKKLPEVIPTAHVISAEGLDDGGDDLHFSSASYRILGERYADKMLELL

SEQ ID NO: 30 - protein, 253 aa, Artificial Sequence
  Unidentified microorganism
VDPKFYIYLCIGQSNMEGQGVIEDCDLSPDERFLMMSTLDCGTRKLGQWYRAIPPLARCDTHLCPADYFGRTMVANLDEGKRVGVVVVAIGGINIDLYDPDGWQSYVGTMNESWQINAVNAYGGNPLGRLLECAREAQKSGVIKGILLHQGENDAYSSVWLQKVKKVYENLLAELNLNAEDVPLIAGEVGNEDQNGICCAANNTINRLPQTIPTAHVVSSVGCTLQSDNLHFDSKGYRKLGRRYAKTMLATMG

SEQ ID NO: 31 - protein, 265 aa, Fibrobacter succinogenes
IYIAYGQSNMAGNGDIVPSEDQAEAPKNFIMLASHNANASQRSGKTNQSIKTGEWYPAIPPMFHPFENLSPADYFGRAMADSLPGVTVGIIPVAIGAVSIRAFDKDQYEAYFRGDGKDIMNWGWPKDYDNNPPGRILELAKKAKEVGVIKGFIFHQGESDGTDANWRKTVYKTYKDVIDALGLDENEVPFVAGELLQEGQNCCSSKNGGIAQLKQNFKKFGLASSKGLQGNGKDPYHFGRAGVIELGKRYCSEMLKLIDKTIDPD

SEQ ID NO: 32 - protein, 235 aa, Rhodopirellula baltica
LFLLAGQSNMAGRGKIADEDLQPHPRVLVFNKAGEWAPAIAPLHFDKPRIAGVGLGRTFAIEYAENNPQATVGLIPCAVGGSSLDVWQPGGFHESTNTHPYDDCMKRMQQAIVAGELKGILWHQGESDSNPALSKTYQSKLNELFERFRTEFGSPNVPIVIGQLGQFTEKPWDESRKLVDQAHRTLPDRMTNTVFVHSDGLGHKGDQTHFSAEAYREFGHRYFLAYQQLTGSSNE

SEQ ID NO: 33 - protein, 232 aa, Solibacter usitatus
IFLLIGQSNMAGRGVVEEQDRQPIPRVFMLNKAMEWVPAIDPVHFDKPDIAGVGLARTFGKVLAAADPNASIGLVPAAFGGTSLEEWKVGGKLYEEAVRRAKFAMSSGKLRGILWHQGEADAGKKELASSYRQRFSAMITQLRADLGEPDVPVVVGQLGEFLSESATPRSPFASVVDEQLATVPLTVPHSAFVSSNGLTSNADHLHFDARSQREFGRRYALAFLSIDASWAH

SEQ ID NO: 34 - protein, 235 aa, Bacteroides thetaiotaomicron
LYLCIGQSNMAGRGKLSPEVMDTLQNVYLLNADDQFEPAVNPLNRYSTIGKGLSWQQVGPAYGFAKTMATKKHPVGLIVNARGGSSIRSWVKNAKQSGGYYDEAIRRAKEAMKYGTLKAIIWHQGEADCHHPEAYKEKIIQLMTDLRNDLGMPDLPVVVGQIAQWNWTKKPYIPEGTKPFNDMIKEISTFLPHSACVSSEGLTPLKDETDPHFDAASQITLGKRYAKEVKKLIKK

SEQ ID NO: 35 - protein, 243 aa, Clostridium acetobutylicum
SFLMLGQSNMAGRGFINEVPMIYNERIQMLRNGRWQMMTEPINYDRPVSGISLAGSFADAWSQKNQEDIIGLIPCAEGGSSIDEWALDGVLFRHALTEAKFAMESSELTGILWHQGESDSLNGNYKVYYKKLLLIIEALRKELNVPDIPIIIGGLGDFLGKERFGKGCTEYNFINKELQKFAFEQDNCYFVTASGLTCNPDGIHIDAISQRKFGLRYFEAFFNRKHVLEPLINENELLNLNYA

SEQ ID NO: 36 - protein, 243 aa, Clostridium beijerinckii
SFLMVGQSNMAGRGFIHEVPQIYNERIQMLRNGRWQMMTEPINYDRHVSGISLAGSFADAWSRQNQEDTIGLIPCAEGGSTLDEWAVDGVLFRHAVTEAKFAMESSELTGILWHQGESDSVNGNYKVYYNKLLLIIEAFRKELNAPDIPIIIGGLGEFLGKEGFGKSCTEYKFINEELQKFAFEQDNCFFVTASGLTSNPDGIHIDAISQRKFGLRYFEAFSNRQHVLKPLINEDELLNLNNA

SEQ ID NO: 37 - protein, 243 aa, Alkaliphilus metalliredigens
SFFMLGQSNMAGRGFIHEVTPIYNERIQMLRNGRWQMMTEPINYDRPVSGISLAASFADAWCLQNQEDTIGLIPCAEGGSSLDEWAVDQALFKHAITEAKFAIQSSELTGILWHQGESDSMNGNYKVYYKKLFLIIEALRKELNAPDIPLIIGGLGDFLGKEGFGISCTEYNFINQELQKFSFEQENCYFVTASGLTSNPDGIHIDAISQRKFGLRYFEAFSNRKHVLEPLIDENELINLNHT

SEQ ID NO: 38 - protein, 243 aa, Clostridium difficile
SFLMLGQSNMAGRGFISEVTPIYNERIQMLRNGRWQMMTEPINYDRPVSGVSLAASFADAWCCENQEDRIGLIPCAEGGSSLDEWNIDGILFKHAISEAKFAIQSSELTGILWHQGENDSNNGNYKFYYKKLLSIIETLRKELNIPDIPIIIGGLGDFLGKVGFGKSCTEYVFINQELQKFAFEQDNCYFVTATGLTSNPDGIHIDAISQRKFGLRYFEAFHKKKHIMEALANESELIIPSNS

SEQ ID NO: 39 - protein, 242 aa, Bacillus amyloliquefaciens
SFLMLGQSNMAGRGFLNEVDPIYNEKIKMLRNGQWQMMTEPINYDRPVSGVGLAASFADAWSKAHPDEEIGLIPCAEGGSSLNDWHPEGILFQHALSETRFALRSSQICGILWHQGESDSYRSLHETYYEKLTLIIGTLRNELKLDEVPLIIGGLGDFLGKTGFGQHATEFRQVNEQLLRFANEQQNCYFVTATGLTANPDGIHLDAASQRKFGYRYFEAFSKKHHILKPISGEEQSLKVNG

SEQ ID NO: 40 - protein, 242 aa, Bacillus licheniformis
SFLMLGQSNMAGRGFLNEVDPIYNEKIKMLRNGQWQMMTEPINYDRPVSGVGLAASFADAWSKAHPDEEIGLIPCAEGGSSLNDWHPEGILFQHALAEARFALRSSQICGILWHQGESDSYRSLHETYYEKLTLIIETLRNELKLDEVPLIIGGLGDFLGKTGFGQHATEFRQVNEQLLRFANEQQNCYFVAAAGLTANPDGIHLDAASQRKFGYRYFEAFSKKYHILKPISGEEQSLKVNG

SEQ ID NO: 41 - protein, 243 aa, Desulfitobacterium hafniense
SFLMIGQSNMAGRGFLNDVPPIYNERIKMLRNGLFQFMEEPINYDRSIAGVGLAASFAAAWCKKNKRDEIGLIPCAEGGSSLDDWSVDDALFANAIAQTKLAQRISTLDGIIWHQGEAESHSGKYRDYYDKFFVIIERLRQVLDVPEIPLIIGGLGDYLGHGIMGGYFNEYSQVNEELKRFAHSHNNCYYVTAEGLTCNPDGIHLNAVSQRIFGLRYYEAYDQKCHILGPVRNENSANEIDND

SEQ ID NO: 42 - protein, 243 aa, Desulfitobacterium hafniense; identical to SEQ ID NO: 41

SEQ ID NO: 43 - protein, 243 aa, Bacillus pumilus
SFLLIGQSNMAGRGFKHEVPPIYNERIMMLRNGRWQMMTEPIHFDRPVAGVGLAASFAETWCKDHEGEKIGLIPCAEGGSSIDEWSRDGALFRHAISEATFAKENSELAGILWHQGESDSQDGKYKEYDEKIRRLFHEIRTELSVPNIPLVIGGLGDFLGKVAFGAGCVEYQLINEELQKYAHRHENCYYVTAKGLIPNPDGIHINAMSQRIFGLRYYEAFRRKQHIHDPLPNEHELVHDCHN

SEQ ID NO: 44 - protein, 243 aa, Bacillus clausi
SILLIGQSNMAGRGFVKDVPPIYNEHIHMLRNGRWQMMAEPLNFDRHVSGIGPAASFAQAWTTDHPGESIGLIPCAEGGSSIDEWTMDSPLTRHAISEATFATETSELIAILWHQGESDSFGERFKTYENKLLSLFTHLREELNVPDIPIIIGELGHYLGERGFGENAVEFKQINQILYKIAHNEENCYFVTSKGLTANPDGIHIDAISQRKFGLRYYEAFSKQKHVLDPLGIEDEWIAKEAK

SEQ ID NO: 45 - protein, 243 aa, Lactobacillus sakei
SILLVGQSNMAGRGFIQDVPGLRHERVKMLRNGRWQMMAEPIHFDREVAGVGPAASFAAAWVQAHPDEELGLIPCAEGGSSIDEWASDEMLMRHAIAEAKFAQESSELIGVLWHQGESDSLKGGYQTYAAKLTAVFSHLRQALGQADLPIIVGQLPDFLGQEGFGASATEFNDINREMANVVAQDPHSYLVNAAELTANPDGIHIDAASQRRFGLRYYAAFANQEDVLAVLPDEAAQLGVLYQ

SEQ ID NO: 46 - protein, 243 aa, Staphylococcus saprophyticus
SILLIGQSNMAGRGFIDEVPPIIDERMMMLRNGKWQMMEEPIHSDRSVAGIGPAASFAKLWLDKHPNETIGLIPCADGGTTIDDWAPDQILTRHALAEATFAQETSEIIGILWHQGESDSLNQRYQDYDKKLKTLINYFREQLNIPEVPFIVGLLPDFLGKAAFGQSAVEYAQINEALKRVTQLTTNSYYVTAQDITANPDAIHINANSQRLLGMRYFAAFSRQTHINQPLPEEKEAETILYK

SEQ ID NO: 47 - protein, 130 aa, Vitis vinifera
MVNRAKESVKSGGEIKALLWYQGESDTSSYNDAKSYKDNMESLIQNVRQDLGSPSLPIIQVAIASGDSKYMERVREAQKEIDFPNVVCVDAKGLPLKEDHLHLTTEAQVRLGQMLADAYLANFAPPPASS

SEQ ID NO: 48 - protein, 130 aa, Vitis vinifera
MVNRAKESVKSGGEIKALLWYQGESDTSSYNDAKSYKDNMESLIQNVRQDLGSPSLPIIQVAIASGDSKYMERVREAQKEIDIPNVVCVDAKGLPLKEDHLHLTTEAQVRLGQMLADAYLANFAPPPASS

SEQ ID NO: 49 - protein, 237 aa, Arabidopsis thaliana
IFILSGQSNMAGRGGVVKDHHHNRWVWDKILPPECAPNSSILRLSADLRWEEAHEPLHVDIDTGKVCGVGPGMAFANAVKNRVETDSAVIGLVPCASGGTAIKEWERGSHLYERMVKRTEESRKCGGEIKAVLWYQGESDVLDIHDAESYGNNMDRLIKNLRHDLNLPSLPIIQVAIASGGGYIDKVREAQLGLKLSNVVCVDAKGLPLKSDNLHLTTEAQVQLGLSLAQAYLSNFC

SEQ ID NO: 50 - protein, 235 aa, Oryza sativa
IFVLSGQSNMAGRGGVHHRRWDGVVPPECAPCPSVLRLTAALDWVEAREPLHADIDTAKTCGVGPGMAFARAVLPRLDPPGSGVGLVPCAVGGTAIREWARGERLYDQMVRRARAAAECGEIEAVLWYQGESDAESDAATAAYAGNLETLIANVREDLGMPQLPFIQVALASGNKKNIEKVRKAQLGINLPNVVTVDAFGLSLNEDHLHLTTESQVKLGEMLAQVYMSNFLPATC

SEQ ID NO: 51 - protein, 235 aa, Oryza sativa
IFVLSGQSNMAGRGGVHHRRWDGVVPPECAPCPSVLRLTAALDWVEAREPLHADIDTAKTCGVGPGMAFARAVLPRLDPPGSGVGLVPCAVGGTAIREWARGERLYDQMVRRARAAAECGEIEAVQWYQGESDAESDAATAAYAGNLETLIANVREDLGMPQLPFIQVALASGNKKNIEKVRKAQLGINLPNVVTVDAFGLSLNEDHLHLTTESQVKLGEMLAQVYMSNFLPATC

SEQ ID NO: 52 - protein, 241 aa, Zea mays
IFVLSGQSNMAGRGGVHHKHWDGVVPPECAPDPSILRLSSAQQWEEAREPLHADIDTTKTCGIGPGMAFARAVLSRLQEDTPGAATQIGIGLVPCAVGGTAIREWSLGKHLYEQMVSRARVATLYGEIEAILWYQGESDAESDADTSAYLENVKRLICNVRADLGMPQLPFIQVALASGNKRNIEKVRNAQFSVNLPNVVTVDPMGMALNEDKLHLTTESQVKLGKMLAEAYILNFSTSTC

SEQ ID NO: 53 - protein, 239 aa, Zea mays
IFVLSGQSNMAGRGGVHHKHWDGVVPPECAPDPSILRLSSAQQWEEAREPLHADIDTTKTCGIGPGMAFARAVLSSLQEDTPGAAAQIGLVPCAVGGTAIREWSLGKHLYEQMVSRARVATLYGEIEAILWYQGESDAESDADTSAYLENVERLICNVRADLGMPQLPFIQVALASGNKRNIEKVRNAQFSVNLPNVVTVDPMGMALNEDKLHLTTESQVKLGKMLAEAYILNFSTSTC

SEQ ID NO: 54 - protein, 233 aa, Oryza sativa
LFLLAGQSNMAGRGALARPLPPPYLPHPRLLRLAASRRWVPAAPPLHADIDTHKTCGLGPAMPFAHRLLLLLHSDEVLGLVPCAVGGTRIWMWARGQPLYEAAIDRARAAVADGGGAIGAVLWFQGESDTIELDDARSYGAKMERLVADLRADLHLPNLLVIQVGLASGEGNYTDIVREAQKNINLPNVLLVDAMGLPLRDDQLHLSTEAQLQLGNMLAEAYLKFNSSRGSML

SEQ ID NO: 55 - protein, 233 aa, Oryza sativa
LFLLAGQYNMAGRGALARPLPPPYLPHPRLLRLTASRRWVTAAPPLHADIDTHKTCGLGPAMPFAHRLLLQTDSEEVLGLVPCAVGGTRIWMWARGQPLYEPAVARARAAVADGGGAIGAVLWFQGESDTIELDDARSYGGKMERLVADLRADLHLPNLLVIQVGLASGEGNYTDIVREAQKNINIPNVLLVDAMGLPLRDDQLHLSTEAQLQLGNMLAEAYLKFNSSRGSML

SEQ ID NO: 56 - protein, 87 aa, Zea mays
MEALVRDVRRDLGMPDLLVIQVGLATGQGRFVDIVREAQRRVSLRNVRYVDAKGLPVANDYTHLTTPAQVKLGNMLAAAYMAATHTH

SEQ ID NO: 57 - protein, 239 aa, Zea mays
VFLLAGQSNMGGRGGATNGTWDGVVPPACAPSPRILRLSPSLRWEEAREPLHAGIDLHNVLGVGPGMPFAHALLRSWRRSGRRPAVVGLVPCAQGATPIASWSRGTPLYDRMLARARAAVARGPATRLAALLWYQGEADTIRRQDADAYTPRMEALVRDVRRDLGMPDLLVIQVGLATGQGRFVDIVREAQRRVSLRNVRYVDAKGLPVANDYTHLTTPAQVKLGNMLAAAYMAATHTH

SEQ ID NO: 58 - protein, 235 aa, Oryza sativa
IFLLGGQSNMGGRGGATNGPWDGVVPPECAPSPRILRLSPELRWEEAREPLHAGIDVHNVLGVGPGMSFAHALFRAIPPSTVIGLVPCAQGGTPIANWTRGTELYERMVGRGRAAMATAGAGAGARMGALLWYQGEADTIRREDAEVYARKMEGMVRDVRRDLALPELLVIQVGIATGQGKFVEPVREAQKAVRLPFLKYVDAKGLPIANDYTHLTTPAQVKLGKLLAKAYLSTL

SEQ ID NO: 59 - protein, 173 aa, Zea mays
MPFAHAVLTSEGAAAEPPVVVGLVPCAQGGTPIANWSRGTELYERMVTRARAAVAECSGRGHLAALLWYQGEADTMRRQDAELYQRRMETLVRDVRCDLGRPDLLVIQVGIATAQYNGKFLGVVREAQKAVKLPNVKYVDAMGLPIASDHTHLTTEAQVQLGNKLAESYLETL

SEQ ID NO: 60 - protein, 236 aa, Zea mays
VFILAGQSNMAGRGGVVANRWDGVVPGDCAPSPAVLRLSPDLRWEEAREPLHAGIDAANHAVGVGPGMAFANALLRSGRAGGAVVGLVPCAVGGTRMAEWGRGTELYAEMLRRARVAVETGGRIGALLWYQGESDTVRWSDATEYGRRMGMLVRDLRADLGIPHLLVIQVGLASGLGQYTQVVRDAQKGIKLRNVRFVDAMGLPLQDGHLHLSTQAQVQLGRMLAQSYLNYGTSQL

SEQ ID NO: 61 - protein, 227 aa, Zea mays
MAGRGGVVANRWDGVVPGDCAPSPAVLRLSPDLRWEEAREPLHAGIDAANHAVGVGPGMAFANALLRSGRAGGAVVGLVPCAVGGTRMAEWGRGTELYAEMLRRARVAVETGGRIGALLWYQGESDTVRWSDATEYGRRMGMLVRDLRADLGIPHLLVIQVGLASGLGQYTQVVRDAQKGIKLRNVRFVDAMGLPLQDGHLHLSTQAQVQLGRMLAQSYLNYGTSQL

SEQ ID NO: 62 - protein, 233 aa, Oryza sativa
VFILGGQSNMAGRGGVVGSHWDGMVPPECAPNPSILRLSPQLRWEEAHEPLHNGIDSNRTCGVGPGMSFANALLRSGQFPVIGLVPCAVGGTRMADWAKGTDLYSDLVRRSRVALETGGRIGAVLWYQGESDTVRWADANEYARRMAMLVRNLRADLAMPHLLLIQVGLASGLGQYTEVVREAQKGIKLRNVRFVDAKGLPLEDGHLHLSTQAQVQLGHMLAQAYLNYGTSTL

SEQ ID NO: 63 - protein, 178 aa, Medicago truncatula
IFILAGQSNMAGRGGVLNGKWDGNIPPECKPNPSILKLNTKLKWEEAHEPLHADIDVGKTCGIGPGLAFANEVVRMSGGECVVGLVPCAVGGTRIEEWRNGSHLYNELVRRSIESVKDGDGVIRAVLWYQGESDTVREEDAERYKYRMENLIENLRLDLQLPSLLVIQCIGFSACQPT

SEQ ID NO: 64 - protein, 178 aa, Medicago truncatula; identical to SEQ ID NO: 63

SEQ ID NO: 65 - protein, 253 aa, Arabidopsis thaliana
IFILAGQSNMAGRGGVYNDTATNTTVWDGVIPPECRSNPSILRLTSKLEWKEAKEPLHVDIDINKTNGVGPGMPFANRVVNRFGQVGLVPCSIGGTKLSQWQKGEFLYEETVKRAKAAMASGGGGSYRAVLWYQGESDTVDMVDASVYKKRLVKFFSDLRNDLQHPNLPIIQVALATGAGPYLDAVRKAQLKTDLENVYCVDARGLPLEPDGLHLTTSSQVQLGHMIAESFLAIPNSARPSLYMHFFSLFCSL

SEQ ID NO: 66 - protein, 230 aa, Clostridium cellulolyticum
CFLLLGQSNMAGYAAAQASDKVEDPRVLVLGYDNNAALGRVTDKWDVACPPLHASWLDAVGPGDWFGKTMIQKVPSGDTIGLIPCAISGEKIETFMKSGGTKYNWIINRAKLAQEKGGVIDGIIFHQGESNSGDPSWPGKVKTLVEDLRKDLNLGNVPFIAGELLYSGPCAGHNTLVNQLPSLITNSYVVSADGLVVDPADTQYRLHFGHDPSVTLGKRYAEKMIQALKW

SEQ ID NO: 67 - protein, 228 aa, Sorangium cellulosum
IFMLMGQSNMAGVAAKQASDQNSDQRLKVLGGCNQPAGQWNLANPPLSDCPGESRINLSTSVDPGIWFGKTLLGKLREGDTIGLIGTAESGESINTFISGGSHHQTILNKIAKAKTAENARFAGIIFHQGETDTGQSSWPGKVVQLYNEMKAAWGVDYDVPFILGELPAGGCCSVHNNLVHQAADMLPDGYWISQEGTKVMDQYHFDHASVVLMGTRYGEKMIEALKW

SEQ ID NO: 68 - protein, 248 aa, Fibrobacter succinogenes
IYIAYGQSNMEGNARNFTDVDKKEHPRVKMFATTSCPSLGRPTVGEMYPAVPPMFKCGEGLSVADWFGRHMADSLPNVTIGIIPVAQGGTSIRLFDPDDYKNYLNSAESWLKNGAKAYGDDGNAMGRIIEVAKKAQEKGVIKGIIFHQGETDGGMSNWEQIVKKTYEYMLKQLGLNAEETPFVAGEMVDGGSCAGFSSRVRGLSKYIANFGVASSKGYGSKGDGLHFTVEGYRGMGLRYAQQMLKLIN

SEQ ID NO: 69 - protein, 264 aa, Cytophaga hutchinsonii
IYLCFGQSNMEGQGPIEAQDQTVDSRFRVMQAVSCTGQPQNAWRTATPPIARCNTKIGPSDYFGREMVKNLPANIKVGIVHVSVAGCKIELFDKTNYNTYLNSLSSADQYIKTTAGQYGGNPYGRLVELAKLAQKDGVIKGILLHQGESNTGDQAWATKVNNVYKNLLADLGLTAANVPLLAGQVVDAAQGGLCASMNTTINALPNTIPTAHVISSSGCTDQSDNLHFTTAGYRLLGTRYAQTMLSLLNITAAAAPVVTLTAPT

SEQ ID NO: 70 - protein, 258 aa, Artificial Sequence
  Unidentified microorganism
IYLCIGQSNMEGQGVIEDCDLSPDERFLMMSTLDCGTRKLGQWYRAIPPLARCDTHLCPADYFGRTMVANLDEGKRVGVVVVAIGGINIDLYDPDGWQSYVGTMNESWQINAVNAYGGNPLGRLLECAREAQKSGVIKGILLHQGENDAYSSVWLQKVKKVYENLLAELNLNAEDVPLIAGEVGNEDQNGICCAANNTINRLPQTIPTAHVVSSVGCTLQSDNLHFDSKGYRKLGRRYAKTMLATMGIEADIDEDEVP

SEQ ID NO: 71 - protein, 258 aa, Neocallimastix patriciarum
IYLAFGQSNMEGQGPIGSQDRTVDKRFQMISTVSGCNGRQMGNWYDAVPPLANCDGKLGPVDYFGRTLVKKLPQEIKVGVAVVAVAGCDIQLFEKNNYRNYRLESYMQGRVNAYGGNPYGRLIEVAKKAQQVGVIKGILLHQGETNTGQQNWPNRVKAVYEDMLKDLGLNAKDVPLLAGEVVQSNQGGQCGSMNSIIQKLPSVIPTAHVISSQGLGQQGDGLHFSSQAYRTFGERYADEMLKILGDVKPVGKTTTTTT

SEQ ID NO: 72 - protein, 244 aa, Orpinomyces sp.
IYLALGQSNMEGQGNVEAQDRVEDKRFKLISTADECMGRELGEWYPALPPIVNCYGNLGPVDYFGRTLTKKLPKEVKVGVCAVAVAGCDIQLFEEENYKSYEIPDWMQGRIDHYGGNPFRRLVNIAKKAQKAGVIKGILLHQGETNNGQEDWPKRIKVVYERLLKELNLKAEEVPLLAGEVVREEYEGMCSLHNTVIKKLPEVIPTAHVISAEGLDDGGDDLHFSSASYRILGERYADKMLELL

SEQ ID NO: 73 - protein, 267 aa, Cytophaga hutchinsonii
IYLTFGQSNMEGNGVIEAQDQTAVNSRFQVMGAVNCTGTKSYTTGKWTTATAPIVRCNTGLGPLDYFGRTMVSNLPANIKVGVVPVAIGGCDIALFDKVNYGSYVATAPSWMIGTINQYGGNPYARLVEVAKLAQKDGVIKGILFHQGETNNGQQDWPAKVKAIYDNLIKDLGLDPAKTPFLAGELVTTAQGGACGGHNSIIAKLPNVIPNAHVVSAAGLPHKGDNLHFTPASYRTFGERYAQLMLTLPAYSNAQTAATNPIINADV

SEQ ID NO: 74 - protein, 239 aa, Artificial Sequence
  Unidentified microorganism
MPAVDYPATDKLPARKMGEWCEAIPPLCRPNTGLTPADWFGRTLVASLPENIKIGVIHVAIGGIDIRGFLPDSIPSYVKRAPNWMKGMLEAYNNNPYERLVTLAKKAQKDGVIKGILMHQGETNTGDPKWAGMVQQVYDHLCGDLQLKPEDVNLYAGNIVQAGGQGVCFACKKQIDELPQTLHTAQVISSDDCSNGPDRLHFDAAGYRELGCRYGEAVARFLGYEPKRPYIEMPKKIEV

SEQ ID NO: 75 - protein, 265 aa, Artificial Sequence
  Unidentified microorganism
IFLCFGQSNMEGNARPEAVDLESPGPRFLLMPAVDFPDKGRKMGEWCEASAPLCRPNTGLTPADWFGRTLVASLPENIKIGVIHVAVGGIKIEGFMPSEIANYVKTEAPGWMKGMLEAYGNNPYERLVTLAKKAQKDGVIKGILMHQGESNTGDPDWAKKVQKVYDSLCSDLKLKPEDVPLFAGNIVQANGQGVCIGCKKQIDELPQTIHTSQVISSDDCSNGPDRLHFDAAGYRELGCRYGEAVARFLGFEPKRPKMPGKKIVV

SEQ ID NO: 76 - protein, 250 aa, Flavobacterium johnsoniae
VYLSFGQSNMEGFAKIEPQDKTGVNERFQVLSAVDCPEMGREKGKWYTAVPPLCRCTTGLTPMDYFGRTMISNLPQNIKVGVVNVAVGGCKIELFDKNNFESYVANSPDWLKNIVKQYDGNPYGRLVEMAKIAQKKGVIKGILLHQGESNTGDTLWPQKVKIVYDNLIKDLNLDPKKVPLLSGETVNEDQNGKCGSMNKIIAKLPQVLPNSYIISSKGCKADADFLHFSPEGYRELGKRYADKMLSLSGY

SEQ ID NO: 77 - protein, 263 aa, Rhodopirellula baltica
VYLLAGQSNMDGRGQVSDLSEEQKQSTGDAIIFYRSVPRESDGWQTLAPGFSVPPKYKGDLPSPTFGPEIGFARSMSNANPNQKLALIKGSKGGTSLRADWKPGVQGDPKSQGPRYRDFIETIRMATKQLSDRGDQFTIRGLLWHQGESDSKSSTERYRRRLEELIVRIREDVGVPDLPVVVGEVFDNGKRDNVRTAIQAVAAASSTVGLVSSEGTTTWDPGTHFDARSQLLLGERYAVAMSELPAPIPATGSSTSVQQRTDR

SEQ ID NO: 78 - protein, 269 aa, Teredinibacter turnerae
IYLMFGQSNMEGQGQISSQDQQVPTGLLAMQADNNCTVGGASYGEWRTATPPLIRCYNTAHAWNNGGLGPGDYFGRTMLENSGAGVRVGLVGAAYQGQSINFFRKNCAALGSCQPSGANGSVPGGAGGYAWMLDLARKAQEDGVIKGIIFHQGESDTGSSTWSSRVNEVVTDLRTDLGLSASEVPFIAGEMVPGACCTSHDARVHEIPSVVANGHYVSAAGLGSRDQYHFNAAGYREIGRRYANKMLELIDVSGSSTSSSSSSSSSSSS

SEQ ID NO: 79 - protein, 252 aa, Roseobacter denitrificans
VFALMGQSNMIGRAAFDGGAKWPDGTLQIGRGGDEDGAIIPARNPADGPATSRPLAHTGARLGNMGLDIQFAIDYLSDKPDVTLLFIPCAQGATGFSNGAWNPGDWLYNRETARINAAMNANPEFLFQGFLWHQGETDTGIPGTYGGLLDNLIAGLRRDVTAATPTTPFILGGLAAGNAGRDAITGIIAATPGRVPYTAFAPTADLSLTDANHFDAASFRTLGARYAAALASAAANQPGAPGIPTGLSAVAG

SEQ ID NO: 80 - DNA, 2337 bp, Fibrobacter succinogenes
atgggattgtttagtaagatggcgaagtcggctgtggttttggctgcttttgctgtagtgaatagtgcagccgttaaggtcaataacccgatcatgtatgtcgatagcccggacccttccattgtccgtgttgacgatgcttattacatggtcacgacgaccatgcactttgcgcctggcgtgcctgttttcaagagcacggatttggcccagtggcgcacggtgggttatgcctaccagacgctcaccaacaacgaccagcagaacctgaatggcggcaaggacgcctacggtaagggctcctgggcatcgagcatccgctaccacaaggggtttttctacgtgctgactccgtcttacacgacgggcaaaactcatttgtacaagaccgccgacgtggaaagcggccagtggtccgaagtgcagctcccgttctaccatgacccttctttgttctttgatgacgatggcaccgtgtgggtgttctacggcagcggcgaccagattagctatgtgcagttgaatgacgatgctagcggtgtaaaggctggcggcaagagcggtaagctcggtggcgtgagtgtaaaccaggtaaccggtaccagcaactactacgtgcagcaggaaggctcgcacatggaaaaggtgaacggcgaatactacctgtttacgatttcctggccggctggcaagagccgtagcgaaatcgtttaccgttccaagagcctgctctccggattcagcggaaggtatttcctctctgataacggtgttgcgcagggcggcatttttgatactcccgatggcaagtggtatgccctcctgttccgcgattccggtccggttggccgtatgtcgcacttggtcccgatggaatggaaggatggttggccggttccgacgagcggatccaaggctccctcgacgattgacttgccggaatctccgctcccgggctacggcatggtcacgagtgacgattttgaatctggcgaacttgctctcgaatggcagttcaaccataaccctgataacaaaaactggagcttgtctgcaaatccgggatttttccgcattaccacgggccgcacggatagccgtgtcgtgaatgcaaagaacactttgacacagcgttcctttggccctaagagttctggccgtacgcttgttgacggcaagggcatgaaggatggagacatggcgggcctcgttgctttgcaggatgacaagggctttgtcgcgcttgctaaagatggcggtaactacaaggttgtgatgtacagcggaaacaaggatggcgaaagacttgtaacaagtgaaaatctttcggactccaaggtttatctgcgaatcgattttgacttgccgattgaccgtggtactgcatatttctattacagcaccgatggtagctcttggaaaaagattggcaacgatgtgaagctcaattatgacctccacatgttcgtgggcgtgcgctggggcctcttcaactttgcgaccaagcaggcgggcggctatgcagacttcgactggttcaaggtcggtaccgatgtgaacgatgaaatttatctcgacggcgctggttctgaacctgttccgcagactccgttctgtgccgctggcgaaaactgccctgcaaatgcaatcccgggcaagattgaagccgaagactttgacgtgccgggcaagggcaaggatggctcatcttactatgacagcgattccgaaaaccacggcgatagcgactatcgcaagggtaccggcgttgacctttacaagaaggcaactggcgtcattgtgggctacaacagcgaaggcgactggctcgaatataccgtgaacgtaaaggaagcgggagattacaccatgtttgctgcggttgctgctgcgggttccacctcaagcttcaagctctctttggatggcaaggacattactgaagaactctctgttccggcggcaagttctggcgaagaaaattatgacgattataacaaggtaaaagcaaacgtaaaactcccggaaggcgaacatgtactccgcttcacggttgttggctcttggcttgatatcgactactttacgttcgtgaagggcgcaaacgctacggatccggatcccattgttggcttggcaaagggcgttcgctataatgtgcagggcgtgcagacttatggcgtctatggcttgaacggtaagttcatcggtcgcgttgatgcatccaacaacttcgatgtgcgtagcaaggtgaacagcctcgtgaaggaaagcggcgtgtacatcgtcaagtctctcaccaccggcaacacccaccgcctatccgtaacaaagtaa

SEQ ID NO: 81 - protein, 778 aa, Fibrobacter succinogenes
MGLFSKMAKSAVVLAAFAVVNSAAVKVNNPIMYVDSPDPSIVRVDDAYYMVTTTMHFAPGVPVFKSTDLAQWRTVGYAYQTLTNNDQQNLNGGKDAYGKGSWASSIRYHKGFFYVLTPSYTTGKTHLYKTADVESGQWSEVQLPFYHDPSLFFDDDGTVWVFYGSGDQISYVQLNDDASGVKAGGKSGKLGGVSVNQVTGTSNYYVQQEGSHMEKVNGEYYLFTISWPAGKSRSEIVYRSKSLLSGFSGRYFLSDNGVAQGGIFDTPDGKWYALLFRDSGPVGRMSHLVPMEWKDGWPVPTSGSKAPSTIDLPESPLPGYGMVTSDDFESGELALEWQFNHNPDNKNWSLSANPGFFRITTGRTDSRVVNAKNTLTQRSFGPKSSGRTLVDGKGMKDGDMAGLVALQDDKGFVALAKDGGNYKVVMYSGNKDGERLVTSENLSDSKVYLRIDFDLPIDRGTAYFYYSTDGSSWKKIGNDVKLNYDLHMFVGVRWGLFNFATKQAGGYADFDWFKVGTDVNDEIYLDGAGSEPVPQTPFCAAGENCPANAIPGKIEAEDFDVPGKGKDGSSYYDSDSENHGDSDYRKGTGVDLYKKATGVIVGYNSEGDWLEYTVNVKEAGDYTMFAAVAAAGSTSSFKLSLDGKDITEELSVPAASSGEENYDDYNKVKANVKLPEGEHVLRFTVVGSWLDIDYFTFVKGANATDPDPIVGLAKGVRYNVQGVQTYGVYGLNGKFIGRVDASNNFDVRSKVNSLVKESGVYIVKSLTTGNTHRLSVTK

SEQ ID NO: 82 - protein, 769 aa, Fibrobacter succinogenes
AHHHHHHVDDDDKMAVKVNNPIMYVDSPDPSIVRVDDAYYMVTTTMHFAPGVPVFKSTDLAQWRTVGYAYQTLTNNDQQNLNGGKDAYGKGSWASSIRYHKGFFYVLTPSYTTGKTHLYKTADVESGQWSEVQLPFYHDPSLFFDDDGTVWVFYGSGDQISYVQLNDDASGVKAGGKSGKLGGVSVNQVTGTSNYYVQQEGSHMEKVNGEYYLFTISWPAGKSRSEIVYRSKSLLSGFSGRYFLSDNGVAQGGIFDTPDGKWYALLFRDSGPVGRMSHLVPMEWKDGWPVPTSGSKAPSTIDLPESPLPGYGMVTSDDFESGELALEWQFNHNPDNKNWSLSANPGFFRITTGRTDSRVVNAKNTLTQRSFGPKSSGRTLVDGKGMKDGDMAGLVALQDDKGFVALAKDGGNYKVVMYSGNKDGERLVTSENLSDSKVYLRIDFDLPIDRGTAYFYYSTDGSSWKKIGNDVKLNYDLHMFVGVRWGLFNFATKQAGGYADFDWFKVGTDVNDEIYLDGAGSEPVPQTPFCAAGENCPANAIPGKIEAEDFDVPGKGKDGSSYYDSDSENHGDSDYRKGTGVDLYKKATGVIVGYNSEGDWLEYTVNVKEAGDYTMFAAVAAAGSTSSFKLSLDGKDITEELSVPAASSGEENYDDYNKVKANVKLPEGEHVLRFTVVGSWLDIDYFTFVKGANATDPDPIVGLAKGVRYNVQGVQTYGVYGLNGKFIGRVDASNNFDVRSKVNSLVKESGVYIVKSLTTGNTHRLSVTK

SEQ ID NO: 83 - protein, 702 aa, Fibrobacter succinogenes
AHHHHHHVDDDDKMAVKVNNPIMYVDSPDPSIVRVDDAYYMVTTTMHFAPGVPVFKSTDLAQWRTVGYAYQTLTNNDQQNLNGGKDAYGKGSWASSIRYHKGFFYVLTPSYTTGKTHLYKTADVESGQWSEVQLPFYHDPSLFFDDDGTVWVFYGSGDQISYVQLNDDASGVKAGGKSGKLGGVSVNQVTGTSNYYVQQEGSHMEKVNGEYYLFTISWPAGKSRSEIVYRSKSLLSGFSGRYFLSDNGVAQGGIFDTPDGKWYALLFRDSGPVGRMSHLVPMEWKDGWPVPTSGSKAPSTIDLPESPLPGYGMVTSDDFESGELALEWQFNHNPDNKNWSLSANPGFFRITTGRTDSRVVNAKNTLTQRSFGPKSSGRTLVDGKGMKDGDMAGLVALQDDKGFVALAKDGGNYKVVMYSGNKDGERLVTSENLSDSKVYLRIDFDLPIDRGTAYFYYSTDGSSWKKIGNDVKLNYDLHMFVGVRWGLFNFATKQAGGYADFDWFKVGTDVNDEIYLDGAGSEPVPQTPFCAAGENCPANAIPGKIEAEDFDVPGKGKDGSSYYDSDSENHGDSDYRKGTGVDLYKKATGVIVGYNSEGDWLEYTVNVKEAGDYTMFAAVAAAGSTSSFKLSLDGKDITEELSVPAASSGEENYDDYNKVKANVKLPEGEHVLRFTVVGSWLDIDYFTFVKGANATDPDPIVG

SEQ ID NO: 84 - protein, 101 aa, Fibrobacter succinogenes
AHHHHHHVDDDDKMGANATDPDPIVGLAKGVRYNVQGVQTYGVYGLNGKFIGRVDASNNFDVRSKVNSLVKESGVYIVKSLTTGNTHRLSVTKTHRLSVTK

(19) United States
(12) Patent Application Publication
(10) Pub. No.: US 2011/0237448 A1
(43) Pub. Date: Sep. 29, 2011

(54) CARBOHYDRATE BINDING MODULE WITH AFFINITY FOR INSOLUBLE XYLAN

(75) Inventors: Shosuke YOSHIDA, Urbana, IL (US); Roderick I. Mackie, Urbana, IL (US); Isaac K. O. Cann, Savoy, IL (US)

(73) Assignee: The Board of Trustees of the University of Illinois, Urbana, IL (US)

(21) Appl. No.: 12/885,270

(22) Filed: Sep. 17, 2010

Related U.S. Application Data
(60) Provisional application No. 61/243,887, filed on Sep. 18, 2009.

(51) Int. Cl. C40B 30/04; C12N 15/63; C12N 5/10; C12N 1/00; C12P 21/02; C07H 21/00; C07K 19/00

(52) U.S. Cl. 506/9; 435/320.1; 435/325; 435/419; 435/243; 435/69.7; 536/23.4; 536/23.2; 530/324; 536/25.3

(57) ABSTRACT

The present disclosure relates to isolated polynucleotides with two polynucleotide sequences linked within one open reading frame, in which the first polynucleotide sequence encodes a peptide that binds to a carbohydrate. The present disclosure also relates to vectors and genetically modified host cells containing such isolated polynucleotides and polypeptides encoded by such isolated polynucleotides. The present disclosure further relates to methods of increasing the ability of a recombinant protein to bind to a carbohydrate and methods of identifying a protein having an ability to bind to a carbohydrate.

### RELATED APPLICATION

This application claims the benefit under 35 USC 119(e) of prior copending U.S. Provisional Patent Application No. 61/243,887, filed Sep. 18, 2009, the disclosure of which is hereby incorporated by reference in its entirety.

### Submission of Sequence Listing on AscII Text File

The content of the following submission on ASCII text file is incorporated herein by reference in its entirety: a computer readable form (CRF) of the Sequence Listing (file name: 658012000400SEQLIST.txt, date recorded: Sep. 17, 2010, size: 147 KB).

### FIELD OF THE INVENTION

The present disclosure relates to methods and compositions for targeting a protein to a carbohydrate.

### BACKGROUND OF THE INVENTION

The development of strategies for biomass conversion to fuels (bio-fuels) is a subject of keen interest in the search for alternative energy resources to fossil-fuels (31). Plant cell matter accounts for 150 to 200 billion tons of biomass on the planet annually (24). It is technically possible, but economically far from realization, to convert plant cell walls to bio-fuels (33). Thus, currently, plant cell wall utilization as a source of bio-fuels is mostly at the laboratory scale, although there is great need to move production to industrial scale.

The main components of plant cell wall are cellulose, hemicellulose, and lignin. These components form complex structures, which provide the plant with physical strength (34). Biologically, there are 2 major steps in the production of alcohols from plant-based feedstocks. The first step is an enzymatic hydrolysis of the plant cell wall components to fermentable sugars, and the second step is fermentation of the resultant sugars into alcohols. A major limitation of the process is the lack of highly efficient biocatalysts required for the first step. However, it is known that microbes that harbor genes encoding enzymes that hydrolyze plant cell wall polysaccharides abound in nature either as individuals or as consortia.

Ruminant animals harbor a variety of plant cell wall degrading bacteria in their first stomach or rumen (19). These animals digest forages with the aid of a microbial consortium that is able to metabolize plant cell wall polysaccharides to short chain fatty acids, the main energy source for the ruminant host. Fibrobacter succinogenes is a ubiquitous rumen bacterium and has been estimated in previous reports to occupy 0.1% to 1.0% of the microbial population in the cattle rumen based on the quantification of 16S rRNA gene as a marker (18, 35). F. succinogenes is a significant cellulolytic rumen bacterium, and it has the ability to grow on crystalline cellulose as a sole source of carbon and energy (10). Additionally, it was demonstrated that this bacterium can solubilize hemicelluloses, although it only partially utilized the constituent monosaccharides released (27). Furthermore, F. succinogenes failed to grow on xylose (26), a constituent of most hemicelluloses. Since F. succinogenes is a highly versatile microbe capable of degrading both cellulose and hemicellulose, the identification and analysis of its polysaccharide-hydrolyzing enzymes are likely to yield more versatile biocatalysts for use in biomass conversion to fuel.

Most polysaccharide-hydrolyzing enzymes have a modular structure with distinct catalytic and carbohydrate-binding modules (Henrissat and Davies, Plant Phys (2000) 124, 1515-1519). This modularity is thought to concentrate and target enzymes to their substrate (Boraston et al., Biochem. J. (2004) 382, 769-781). Maintaining the association of a hydrolyzing enzyme with its carbohydrate substrate is critical for increasing the efficiency and speed of catalysis. Although many carbohydrate-binding modules from various enzymes have been studied, those modules contained in polysaccharide-hydrolyzing enzymes from versatile microbes such as rumen bacteria remain to be fully explored and analyzed. Thus, a need exists for identifying additional carbohydrate-binding modules with diverse substrate specificities.

### BRIEF SUMMARY OF THE INVENTION

In order to meet this need, the present disclosure describes isolated polynucleotides containing a carbohydrate binding module and methods of increasing the ability of a recombinant protein to bind to a carbohydrate.

Thus one aspect includes isolated polynucleotides containing a first polynucleotide sequence that encodes SEQ ID NO: 1 wherein said first polynucleotide is linked within one open reading frame to a second polynucleotide sequence to form a linked polynucleotide, wherein SEQ ID NO: 1 binds to a carbohydrate and wherein the linked polynucleotide does not encode a naturally occurring polypeptide. Naturally occurring polypeptides are peptides that occur in nature. In certain embodiments, the first polynucleotide sequence is located within the second polynucleotide sequence. In other embodiments, the first polynucleotide sequence is located at one end of the second polynucleotide sequence. In certain embodiments, the first polynucleotide sequence is separated from the second polynucleotide sequence by a polynucleotide encoding a linker. In certain embodiments, the isolated polynucleotide includes multiple copies of the first polynucleotide sequence. In certain embodiments, the second polynucleotide sequence encodes a peptide. In certain embodiments, the peptide includes a secretion signal. In certain embodiments, the peptide includes a membrane-spanning domain. In certain embodiments, the peptide includes a cell attachment peptide. In certain embodiments, the peptide comprises SEQ ID NO: 1. In certain embodiments, the peptide includes a carbohydrate-binding module (CBM). In certain embodiments, the second polynucleotide sequence encodes a polypeptide. In certain embodiments, the polypeptide includes an enzyme. In certain embodiments, the enzyme is a carbohydrate-active enzyme. In certain embodiments, the carbohydrate-active enzyme has increased enzymatic activity compared to a polypeptide encoding the carbohydrate-active enzyme that is not linked to the first polynucleotide sequence. In certain embodiments, the polypeptide includes an immunoglobulin. In certain embodiments, the polypeptide includes a cytokine. In certain embodiments, the polypeptide includes an endogenous domain having the amino acid sequence of SEQ ID NO: 1. In certain embodiments, binding of the polypeptide to a carbohydrate is increased compared to a polypeptide that is not linked to the first polynucleotide sequence. In certain embodiments, the carbohydrate is insoluble in water. In certain embodiments, the carbohydrate comprises hemicellulose. In certain embodiments, the hemicellulose includes xylan. In certain embodiments, secretion of the polypeptide by a cell is increased compared to a polypeptide that is not linked to the first polynucleotide sequence. In certain embodiments, expression of the polypeptide in a cell is increased compared to a polypeptide that is not linked to the first polynucleotide sequence. In certain embodiments, the polypeptide is more resistant to digestion by proteases compared to a polypeptide that is not linked to the first polynucleotide sequence. In certain embodiments, the second polynucleotide sequence encodes a protein tag. In certain embodiments, the protein tag is selected from the group consisting of a Myc tag, a His tag, a maltose binding protein tag, a glutathione-S-transferase tag, an HA tag, a FLAG tag, and a Green fluorescent protein tag.

Another aspect includes vectors containing the isolated polynucleotide of the previous aspect. Another aspect includes genetically modified host cells containing the vector of the previous aspect.

Yet another aspect includes recombinant polypeptides containing the amino acid sequence encoded by the isolated polynucleotide of the previous aspect. Another aspect includes isolated polypeptides containing SEQ ID NO: 1 conjugated to an atom or a molecule. In certain embodiments, the atom or molecule is selected from one or more of the group of a fluorophore, a radionuclide, a toxin, a polymer, a fragrance particle, a small molecule, a polypeptide, and a peptide.

Another aspect includes methods of increasing the ability of a recombinant protein to bind to a carbohydrate, including the steps of linking a first isolated polynucleotide encoding SEQ ID NO: 1 to a second isolated polynucleotide encoding a polypeptide, a peptide, or a protein tag to form a linked polynucleotide, wherein the linked polynucleotide encodes a recombinant protein having an increased ability to bind to a carbohydrate compared to the polypeptide, peptide, or protein tag alone. In certain embodiments the methods further include the step of expressing the linked polynucleotides in a host cell, wherein expression of the polynucleotides produces the recombinant protein. In certain embodiments, the host cell includes a cell wall and the recombinant protein binds a carbohydrate component of the cell wall. In certain embodiments, the methods further include the step of isolating the carbohydrate-bound recombinant protein. In certain embodiments, the methods further include the step of contacting the host cell with the carbohydrate. In certain embodiments, the second isolated polynucleotide encodes a polypeptide containing a domain selected from one or more of the group of a secretion signal domain and a membrane spanning domain. In certain embodiments, the methods further include the step of contacting the recombinant protein with the carbohydrate. In certain embodiments, the methods further include the step of isolating the carbohydrate-bound recombinant protein. In certain embodiments, the methods further include the step of contacting the carbohydrate-bound recombinant protein with a plurality of cells. In certain embodiments, the second isolated polynucleotide encodes a cell-attachment peptide. In certain embodiments, the second isolated polynucleotide encodes an immunoglobulin. In certain embodiments, the methods further include the step of testing the recombinant protein for its ability to act on the carbohydrate, wherein testing comprises assaying for degradation, modification, or creation of glycosidic bonds on the carbohydrate. In certain embodiments, the carbohydrate is insoluble. In certain embodiments, the carbohydrate includes hemicellulose. In certain embodiments, the hemicellulose includes xylan. In certain embodiments, the methods further include the step of detecting the carbohydrate-bound recombinant protein by incubating the carbohydrate-bound recombinant protein with an antibody specific to the polypeptide, peptide, or protein tag.

Another aspect includes methods of increasing the ability of a recombinant protein to bind to a carbohydrate, including the steps of linking a first isolated polynucleotide encoding SEQ ID NO: 1 to a second isolated polynucleotide encoding an amino acid sequence selected from a library of amino acid sequences to form a linked polynucleotide, wherein the linked polynucleotide encodes a recombinant protein having an increased ability to bind to a carbohydrate compared to the amino acid sequence alone.

Yet another aspect includes methods of identifying a protein having an ability to bind a carbohydrate, including the steps of providing a labeled polynucleotide, wherein the polynucleotide encodes SEQ ID NO: 1, hybridizing the labeled polynucleotide to a homologous sequence in a nucleotide library, and isolating the sequence bound by the labeled polynucleotide, wherein the sequence encodes a protein having an ability to bind to a carbohydrate. In certain embodiments, the nucleotide library is a cDNA library. In certain embodiments, the nucleotide library is a genomic library.

### BRIEF DESCRIPTION OF THE DRAWINGS

FIG. 1 shows the domain architectures of proteins harboring Fibrobacter succinogenes-specific domain-1 (FPd-1) in Fibrobacter succinogenes S85. Proteins harboring FPd-1 domain were obtained through a search of the genome database of Fibrobacter succinogenes S85. The presence of signal peptide was determined by LipoP server and marked as star shapes at the N-terminus of protein architectures. Domain organizations were predicted using BLAST protein searches.

FIG. 2 shows truncational mutant proteins of FSUAxe6B. (A) Schematic representation of translated FSUAxe6B, the mature protein (WT) and its truncational mutant proteins (TM1 to TM5). The DNA primer sequences used for the amplification of the genes are described in Table 1. The arrowheads display the 5′→3′ direction of oligonucleotides. (B) SDS-PAGE image of purified WT and truncational proteins. Purified protein (2.5 μg) was loaded on a 12.5% polyacrylamide gel and stained with Coomassie Brilliant Blue G-250.

FIG. 3 shows qualitative polysaccharide binding studies of FSUAxe6B wild-type (WT) and its truncational mutants. Insoluble oat-spelt xylan (is-OSX) or Avicel PH-101 (Avc) was incubated with 2 μM protein. Lane P represents the same amount of protein incubated in the same buffer, but without substrate. The supernatants after incubation of the proteins with is-OSX or Avc were loaded on SDS-PAGE as (P+OSX) and (P+Avc), respectively. In each case, except for TM5, 10 μL of solution with or without substrate for WT, TM1, TM2, TM3, and TM4 were loaded for the SDS-PAGE analysis. The supernatants of TM5 protein were concentrated up to 10 times, and then 10 μL of the solution was loaded on SDS-PAGE for visualization.

FIG. 4 shows quantitative studies on the binding of FSUAxe6B wild-type (WT) and its truncational mutants to insoluble oat-spelt xylan (is-OSX). Is-OSX (20 mg) was mixed with various concentrations of proteins, and the binding activities were estimated as described in Example 4. The graphs depict the binding isotherms between bound proteins (nmol/g of is-OSX) and free proteins (μM). Panel (A) shows the binding isotherms (closed triangles) for WT and TM1, the protein with the FPd-1 deleted (open triangles). The binding isotherms for TM3 (open squares), TM4 (grey squares), TM5 (closed squares) are shown in panel B. The binding constants for the wild type and its truncated mutants are presented in Table 3.

FIG. 5, part A, shows a multiple amino acid sequence alignment among FPd-1 domains in Fibrobacter succinogenes S85. Amino acid sequences of FPd-1 homologs in Fibrobacter succinogenes S85 were aligned utilizing ClustalW. The output files were entered into the BoxShade ver. 3.21 program (available at www.ch.embnet.org/software/BOX_form.html), with the fraction of sequences that must agree for shading set at 0.5. The conserved amino acids were shaded black, and similar amino acids were shaded gray. The pis of the FPd-1 peptides are shown with protein IDs. Aromatic residues are indicated with arrows. FIG. 5, part B, shows qualitative polysaccharide binding studies of TM5 and its site-directed alanine mutants with insoluble oat-spelt xylan. FSUAxe6B (SEQ ID NO: 2), FSU2266 (SEQ ID NO: 3), FSU2263 (SEQ ID NO: 4), FSU2262 (SEQ ID NO: 5), FSU2292 (SEQ ID NO: 6), FSU2294 (SEQ ID NO: 7), FSU2293 (SEQ ID NO: 8), FSU2851 (SEQ ID NO: 9), FSU2288 (SEQ ID NO: 10), FSU2265 (SEQ ID NO: 11), FSU3103 (SEQ ID NO: 12), FSU2269 (SEQ ID NO: 13), FSU3006 (SEQ ID NO: 14), FSU0777 (SEQ ID NO: 15), FSU2741 (SEQ ID NO: 16), FSU2272 (SEQ ID NO: 17), FSU2274 (SEQ ID NO: 18), FSU2270 (SEQ ID NO: 19), FSU2264 (SEQ ID NO: 20), FSU2516 (SEQ ID NO: 21), FSU0053 (SEQ ID NO: 22), FSU0192 (SEQ ID NO: 23), FSU3053 (SEQ ID NO: 24), FSU3135 (SEQ ID NO: 25).

FIG. 6 shows an amino acid sequence alignment of the FSUAxe6B (SEQ ID NO: 26) esterase domain and similar domains from Carbohydrate Esterase family 6 (CE6) proteins. Amino acid sequences of FSUAxe6B and biochemically characterized CE6 proteins from Fibrobacter succinogenes (GenBank accession no. AAG36766; SEQ ID NO: 27), Neocallimastix patriciarum (Genbank accession no. AAB69090; SEQ ID NO: 28), Orpinomyces sp. PC-2 (Genbank accession no. AAC14690; SEQ ID NO: 29), and an unidentified microorganism (Genbank accession no. CAJ19130; SEQ ID NO: 30) were aligned utilizing ClustalW. The output files were entered into the BoxShade ver. 3.21 program (available at www.ch.embnet.org/software/BOX_form.html), with the fraction of sequences that must agree for shading set at 1.0. The conserved amino acids were shaded black, and similar amino acids were shaded gray. Arrowheads indicate the catalytic residues identified in this study for FSUAxe6B. An expanded alignment is shown in FIG. 8.

FIG. 7 shows active site residues of FSUAxe6B. (A) Predicted reaction mechanism of FSUAxe6B. The two residues E194 and D270 form hydrogen bonds (dotted lines) with H273, leading to an increase in the pKₐ of its imidazole nitrogen. H273 acts as a strong general base and removes a proton from the hydroxyl group of serine. The deprotonated serine serves as a nucleophile and attacks the carbonyl carbon of the acetyl group. (B) The 3-D structure illustrating the predicted active site residues of FSUAxe6B (FIG. 7A) in a putative acetylxylan esterase from Clostridium acetobutylicum (PDB number; 1ZMB). The side chains in the C. acetobutylicum protein are presented in the model. The corresponding residues in FSUAxe6B are shown in blue letters in closed brackets.

FIG. 8 shows a multiple amino acid sequence alignment of biochemically characterized and putative CE6 proteins. The sequences belonging to CE6 proteins were obtained from the CAZy database (available at www.cazy.org), and aligned with that of FSUAxe6B (SEQ ID NO: 31) utilizing ClustalW. The Genbank accession numbers (source of organism) are as follows: CAD78234 (Rhodopirellula Baltica SH 1; SEQ ID NO: 32), ABJ86882 (Solibacter usitatus E11in6076; SEQ ID NO: 33), AA079285 (Bacteroides thetaiotaomicron VPI-5482; SEQ ID NO: 34), AAK78508 (Clostridium acetobutylicum ATCC 824; SEQ ID NO: 35), ABR35716 (Clostridium beijerinckii NCIMB 8052; SEQ ID NO: 36), ABR50009 (Alkaliphilus metalliredigens QYMF; SEQ ID NO: 37), CAJ68761 (Clostridium difficile 630; SEQ ID NO: 38), ABS74765 (Bacillus amyloliquefaciens FZB42; SEQ ID NO: 39), AAU41672 (Bacillus licheniformis DSM 13; SEQ ID NO: 40), ACL19645 (Desulfitobacterium hafniense DCB-2; SEQ ID NO: 41), BAE85542 (Desulfitobacterium hafniense Y51; SEQ ID NO: 42), ABV61814 (Bacillus pumilus SAFR-032; SEQ ID NO: 43), BAD63143 (Bacillus clausii KSM-K16; SEQ ID NO: 44), CAI54447 (Lactobacillus sakei 23K; SEQ ID NO: 45), BAE19338 (Staphylococcus saprophyticus ATCC 15305; SEQ ID NO: 46), CAN82802 (Vitis vinifera; SEQ ID NO: 47), CAN66317 (Vitis vinifera; SEQ ID NO: 49, AAM65927 (Arabidopsis thaliana; SEQ ID NO:49), CAH67955 (Oryza sativa Indica Group; SEQ ID NO: 50), CAE05089 (Oryza sativa Japonica Group; SEQ ID NO: 51), ACG24977 (Zea mays; SEQ ID NO: 52), ACG48250 (Zea mays; SEQ ID NO: 53), CAH67782 (Oryza sativa Indica Group; SEQ ID NO: 54), CAD39440 (Oryza sativa Japonica Group; SEQ ID NO: 55), ACF83847 (Zea mays B73; SEQ ID NO: 56), ACG40932 (Zea mays; SEQ ID NO: 57), AAP21390 (Oryza sativa Japonica Group; SEQ ID NO: 58), ACG35438 (Zea mays; SEQ ID NO: 59), ACF85252 (Zea mays B73; SEQ ID NO: 60), ACF82807 (Zea mays B73; SEQ ID NO: 61), AAP21393 (Oryza sativa Japonica Group; SEQ ID NO: 62), ABD33289 (Medicago truncatula; SEQ ID NO: 63), ABD32611 (Medicago truncatula; SEQ ID NO: 64), BAF01263 (Arabidopsis thaliana; SEQ ID NO: 65), ACL75596 (Clostridium cellulolyticum H10; SEQ ID NO: 66), CAN99484 (Sorangium cellulosum ‘So ce 56’; SEQ ID NO: 67), AAG36766 (Fibrobacter succinogenes S85; SEQ ID NO: 68), ABG58511 (Cytophaga hutchinsonii ATCC 33406; SEQ ID NO: 69), (CAJ19130 unidentified microorganism; SEQ ID NO: 70), AAB69090 (Neocallimastix patriciarum; SEQ ID NO: 71), AAC14690 (Orpinomyces sp. PC-2; SEQ ID NO: 72), ABG59304 (Cytophaga hutchinsonii ATCC 33406; SEQ ID NO: 73), CAJ19122 (unidentified microorganism; SEQ ID NO: 74), CAJ19109 (unidentified microorganism; SEQ ID NO: 75), ABQ06889 (Flavobacterium johnsoniae UW101; SEQ ID NO: 76), CAD71736 (Rhodopirellula Baltica SH 1; SEQ ID NO: 77), ACR11748 (Teredinibacter turnerae T7901; SEQ ID NO: 78), and Roseobacter denitrificans OCh 114 (ABI93412; SEQ ID NO: 79). The output files were visually inspected, and manual corrections were carried out. The resultant files were shaded with BoxShade ver. 3.21 program (available at www.ch.embnet.org/software/BOX_form.html). Conserved and similar residues were shaded black and grey, respectively. The fraction of sequences that must agree for shading was set at 0.5. Arrowheads indicate the catalytic residues demonstrated in this study to be involved in the catalytic activity (catalytic tetrad) of FSUAxe6B.

FIG. 9 shows qualitative binding assays of FSUAxe6B FPd-1 (TM5) for Avicel and insoluble oat-spelt xylan (is-OSX).

FIG. 10 shows isothermal titration calorimetric (ITC) analysis for FBD-1 (TM5) protein. Part A shows the positive control. Part B shows TM5 vs. arabinoxylan. Part C shows TM5 vs. xylobiose. Part D shows TM5 vs. xylopentaose.

FIG. 11 shows the nucleotide and amino acid sequences of FSU2269 (parts A (SEQ ID NO: 80) and B (SEQ ID NO: 81)) and its protein domain organization (part C).

FIG. 12, part A, shows purified FSU2269 protein on SDS-PAGE. Part B shows an illustration of β-1,4-xylan. Part C shows an α-L-arabinofuranosidase activity assay for FSU2269.

FIG. 13, part A, shows the domain organization of wild-type (WT) and truncational mutants of FSU2269. Part B shows their amino acid sequences: recombinant FSU2269 WT protein (SEQ ID NO: 82), recombinant FSU2269 TM protein (SEQ ID NO: 83), and recombinant FSU2269 FPd-1 protein (SEQ ID NO: 84). Part C shows an α-L-arabinofuranosidase activity assay for FSU2269 WT and TM proteins.

FIG. 14 shows qualitative binding assays of FSU2269 FPd-1 for Avicel or insoluble oat-spelt xylan.

FIG. 15A shows the amino acid sequences of FPd-1 domains for various F. succinogenes proteins. FIG. 15B shows the alignment of those sequences in order to generate a consensus sequence. FSUAxe6B (SEQ ID NO: 2), FSU2266 (SEQ ID NO: 3), FSU2263 (SEQ ID NO: 4), FSU2262 (SEQ ID NO: 5), FSU2292 (SEQ ID NO: 6), FSU2294 (SEQ ID NO: 7), FSU2293 (SEQ ID NO: 8), FSU2851 (SEQ ID NO: 9), FSU2288 (SEQ ID NO: 10), FSU2265 (SEQ ID NO: 11), FSU3103 (SEQ ID NO: 12), FSU2269 (SEQ ID NO: 13), FSU3006 (SEQ ID NO: 14), FSU0777 (SEQ ID NO: 15), FSU2741 (SEQ ID NO: 16), FSU2272 (SEQ ID NO: 17), FSU2274 (SEQ ID NO: 18), FSU2270 (SEQ ID NO: 19), FSU2264 (SEQ ID NO: 20), FSU2516 (SEQ ID NO: 21), FSU0053 (SEQ ID NO: 22), FSU0192 (SEQ ID NO: 23), FSU3053 (SEQ ID NO: 24), FSU3135 (SEQ ID NO: 25).

FIG. 16 shows a list of FPd-1-containing proteins for analysis of binding properties.

FIG. 17 shows qualitative binding assays of FPd-1 peptides for Avicel or insoluble oat-spelt xylan (is-OSX). Consensus (SEQ ID NO: 1), FSUAxe6B (SEQ ID NO: 2), FSU2266 (SEQ ID NO: 3), FSU2263 (SEQ ID NO: 4), FSU2262 (SEQ ID NO: 5), FSU2292 (SEQ ID NO: 6), FSU2294 (SEQ ID NO: 7), FSU2293 (SEQ ID NO: 8), FSU2851 (SEQ ID NO: 9), FSU2288 (SEQ ID NO: 10), FSU2265 (SEQ ID NO: 11), FSU3103 (SEQ ID NO: 12), FSU2269 (SEQ ID NO: 13), FSU3006 (SEQ ID NO: 14), FSU0777 (SEQ ID NO: 15), FSU2741 (SEQ ID NO: 16), FSU2272 (SEQ ID NO: 17), FSU2274 (SEQ ID NO: 18), FSU2270 (SEQ ID NO: 19), FSU2264 (SEQ ID NO: 20), FSU2516 (SEQ ID NO: 21), FSU0053 (SEQ ID NO: 22), FSU0192 (SEQ ID NO: 23), FSU3053 (SEQ ID NO: 24), FSU3135 (SEQ ID NO: 25).

### DETAILED DESCRIPTION OF THE INVENTION

The present disclosure relates to isolated polynucleotides containing two polynucleotide sequences linked within one open reading frame, in which the first polynucleotide sequence encodes a peptide that binds to a carbohydrate. The present disclosure also relates to vectors and genetically modified host cells containing such isolated polynucleotides and polypeptides encoded by such isolated polynucleotides. The present disclosure further relates to methods of increasing the ability of a recombinant protein to bind to a carbohydrate and methods of identifying a protein having an ability to bind to a carbohydrate. The methods include linking a first isolated polynucleotide encoding a peptide that binds to a carbohydrate to a second isolate polynucleotide that encodes a polypeptide, a peptide, or a protein tag.

Polynucleotides of the Invention

The invention herein relates to isolated polynucleotides containing a first polynucleotide sequence linked within one open reading frame to a second polynucleotide sequence, in which the first polynucleotide sequence encodes a carbohydrate binding module.

As used herein, the terms “polynucleotide,” “nucleic acid sequence,” “sequence of nucleic acids,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing nonnucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog; internucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters); those containing pendant moieties, such as, for example, proteins (including nucleases, toxins, antibodies, signal peptides, poly-L-lysine, etc.); those with intercalators (e.g., acridine, psoralen, etc.); and those containing chelators (e.g., metals, radioactive metals, boron, oxidative metals, etc.). As used herein, the symbols for nucleotides and polynucleotides are those recommended by the IUPAC-IUB Commission of Biochemical Nomenclature (Biochem. 9:4022, 1970).

As used herein, the term “open reading frame” or “ORF” is a possible translational reading frame of DNA or RNA (e.g., of a gene), which is capable of being translated into a polypeptide. That is, the reading frame is not interrupted by stop codons. However, it should be noted that the term ORF does not necessarily indicate that the polynucleotide is, in fact, translated into a polypeptide. In preferred embodiments of the invention, the linked polynucleotides do not encode a naturally occurring polypeptide. A naturally occurring polypeptide is a polypeptide that exists in nature without the intervention of humans.

The first polynucleotide sequence encodes SEQ ID NO: 1, a carbohydrate binding module. The sequence of SEQ ID NO: 1 is as follows,

aaxxxaxaxx------xaxxxYxVFDaxGbbLGxaxAxx----caxxa-
--abxxaxxb----GVYaVRxxxxsxxxbVxVxc--.

“a” may be any aliphatic amino acid residue. Aliphatic residues include, for example, isoleucine, valine, and leucine. “b” may be any basic amino acid residue. Basic residues include, for example, arginine, lysine, and histidine. “c” may be any charged amino acid residue. “s” may be any small amino acid residue. Charged residues include, for example, the basic residues as listed above plus aspartate and glutamate. “x” may be any amino acid residue. “-” indicates that this position may contain any amino acid residue or contain no amino acid residue. Any amino acid residue designated as “F” (phenylalanine) or “Y” (tyrosine) in SEQ ID NO: 1 may be substituted with any other aromatic residue. Aromatic residues include, for example, phenylalanine, tyrosine, tryptophan, and histidine. Any amino acid residue designated in SEQ ID NO: 1 as “A” (alanine), “L” (leucine), or “V” (valine) may be substituted with any other aliphatic residue. Any amino acid residue designated in SEQ ID NO: 1 as “R” (arginine) may be substituted with any other basic residue.

SEQ ID NO: 1 can be found, for example, in carbohydrate-active enzymes from F. succinogenes such as FSUAxe6B, FSU2266, FSU2263, FSU2262, FSU2292, FSU2294, FSU2293, FSU2851, FSU2288, FSU2265, FSU3103, FSU2269, FSU3006, FSU0777, FSU2741, FSU2272, FSU2274, FSU2270, FSU2264, FSU2516, FSU0053, FSU0192, FSU3053, and FSU3135.

The linked polynucleotides may be arranged in any way within the open reading frame as long as the arrangement does not interfere with translation of the polynucleotides. For example, the first polynucleotide may be located within the second polynucleotide or at one end of the second polynucleotide. The first and second polynucleotides may be separated by a polynucleotide encoding a linker. A linker may be any amino acid sequence that connects the amino acid sequences encoded by the first and second polynucleotides. In some embodiments, the isolated polynucleotide may comprise multiple copies of the first polynucleotide.

In certain embodiments of the invention, the second polynucleotide encodes a peptide. As used herein, a “peptide” is an amino acid sequence containing a plurality of consecutive polymerized amino acid residues, generally of a length that is less than 30-50 amino acid residues in length and preferably about 2 to 30 amino acid residues in length. The peptide optionally comprises modified amino acid residues, naturally occurring amino acid residues not encoded by a codon, or non-naturally occurring amino acid residues. The peptide may comprise, for example, a secretion signal, a membrane-spanning domain, a cell attachment peptide, the sequence of SEQ ID NO: 1, or a carbohydrate-binding module. A secretion signal directs proteins from the cytosol to the endoplasmic reticulum and, ultimately, to be secreted by the cell. A membrane-spanning domain is a hydrophobic domain that anchors a protein within the cell membrane. A cell attachment peptide promotes attachment of a protein to a cell surface. A carbohydrate-binding module (CBM) is a contiguous amino acid sequence found within a carbohydrate-active enzyme with a discreet fold having carbohydrate-binding activity (Boraston et al., Biochem. J. (2004) 382, 769-781). A few exceptions are CBMs found in cellulosomal scaffoldin proteins and rare instances of independent putative CBMs.

In certain embodiments of the invention, the second polynucleotide encodes a polypeptide. As used herein, a “polypeptide” is an amino acid sequence containing a plurality of consecutive polymerized amino acid residues e.g., at least about 15 consecutive polymerized amino acid residues, optionally at least about 30 consecutive polymerized amino acid residues, or at least about 50 consecutive polymerized amino acid residues. The polypeptide optionally comprises modified amino acid residues, naturally occurring amino acid residues not encoded by a codon, or non-naturally occurring amino acid residues. As used herein, “protein” refers to an amino acid sequence, oligopeptide, peptide, polypeptide, or portions thereof whether naturally occurring or synthetic.

In some instances, the polypeptide comprises an enzyme. In preferred embodiments in which the polypeptide comprises an enzyme, the enzyme is a carbohydrate-active enzyme. As used herein, a “carbohydrate-active enzyme” is any enzyme that can degrade, modify, or create glycosidic bonds. Carbohydrate-active enzymes include, for example, glycoside hydrolases, glycosyltransferases, polysaccharide lyases, and carbohydrate esterases (Cantarel et al. Nucleic Acids Research (2009) 37, D233-D238). In certain embodiments, a carbohydrate-active enzyme that is linked to SEQ ID NO: 1 has increased enzymatic activity compared to a carbohydrate-active enzyme that is not linked to the first polynucleotide sequence.

In other instances, the polypeptide may comprise, for example, an immunoglobulin, a cytokine, or an endogenous domain having the amino acid sequence of SEQ ID NO: 1. An immunoglobulin or antibody provides tight and specific binding to any antigen for which an antibody exists or to which an antibody can be made. A cytokine is a signaling molecule used in cellular communication. An “endogenous domain” as used herein refers to an amino acid sequence or the nucleotide sequence encoding such an amino acid sequence that occurs naturally in a polypeptide and was not introduced into the polypeptide using recombinant engineering techniques. For example, the term refers to a domain that was present in the polypeptide when it was originally isolated from nature.

In preferred embodiments of the invention, binding of a polypeptide that is linked to SEQ ID NO: 1 to a carbohydrate is increased compared to a polypeptide that is not linked to SEQ ID NO: 1. In preferred embodiments, binding is to an insoluble carbohydrate. In other preferred embodiments, binding is to a carbohydrate containing hemicellulose. Hemicellulose is a polymer of short, highly-branched chains of mostly five-carbon pentose sugars (e.g. xylose and arabinose) and to a lesser extent six-carbon hexose sugars (e.g. galactose, glucose and mannose). Hemicelluloses may comprise, for example, xylan, glucuronoxylan, arabinoxylan, glucomannan, or xyloglucan. Non-limiting examples of sources of carbohydrates include grasses (e.g., switchgrass, Miscanthus), rice hulls, bagasse, cotton, jute, hemp, flax, bamboo, sisal, abaca, straw, leaves, grass clippings, corn stover, corn cobs, distillers grains, legume plants, sorghum, sugar cane, sugar beet pulp, wood chips, sawdust, and biomass crops (e.g., Crambe).

Certain desirable properties of the polypeptide may be enhanced when it is linked to SEQ ID NO: 1. For example, secretion of the polypeptide by a cell may be increased, expression of the polypeptide in a cell may be increased, or resistance of the polypeptide to digestion by proteases may be increased.

In certain embodiments of the invention, the second polynucleotide encodes a protein tag. The term “protein tag” refers to an amino acid, peptide or protein that when added to another sequence, provides additional utility or confers useful properties, particularly in the detection or isolation of that sequence. Protein tags may be useful for affinity purification, solubilization, providing epitopes for recognition by antibodies, and detection by fluorescence. Protein tags include, for example, a Myc tag, a His tag, maltose binding protein (MBP), glutathione-S-transferase (GST), HA, FLAG, GFP, or any other protein tags known to one of skill in the art.

Vectors of the Invention

The invention herein relates to vectors containing isolated polynucleotides containing a first polynucleotide sequence encoding SEQ ID NO: 1 linked within one open reading frame to a second polynucleotide sequence.

In preferred embodiments of the invention, the vector is any vector that allows for expression of the linked polynucleotides in a host cell. A typical expression vector contains the desired polynucleotide preceded by one or more regulatory regions, along with a ribosome binding site, e.g., a nucleotide sequence that is 3-9 nucleotides in length and located 3-11 nucleotides upstream of the initiation codon in E. coli. See Shine et al. (1975) Nature 254:34 and Steitz, in Biological Regulation and Development: Gene Expression (ed. R. F. Goldberger), vol. 1, p. 349, 1979, Plenum Publishing, N. Y.

Regulatory regions include, for example, those regions that contain a promoter and an operator. A promoter is operably linked to the desired polynucleotide, thereby initiating transcription of the polynucleotide via an RNA polymerase enzyme. The term “operably linked” as used herein refers to a configuration in which a control sequence is placed at an appropriate position relative to the coding sequence of the DNA sequence or polynucleotide such that the control sequence directs the expression of a polypeptide. An operator is a sequence of nucleic acids adjacent to the promoter, which contains a protein- binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. Examples include lactose promoters (Lad repressor protein changes conformation when contacted with lactose, thereby preventing the Lad repressor protein from binding to the operator) and tryptophan promoters (when complexed with tryptophan, TrpR repressor protein has a conformation that binds the operator; in the absence of tryptophan, the TrpR repressor protein has a conformation that does not bind to the operator). Another example is the tac promoter. (See deBoer et al. (1983) Proc. Natl. Acad. ScL USA, 80:21-25.) As will be appreciated by those of ordinary skill in the art, these and other expression vectors may be used in the present invention, and the invention is not limited in this respect.

Although any suitable expression vector may be used to incorporate the desired sequences, readily available expression vectors include, without limitation: plasmids, such as pSC101, pBR322, pBBR1MCS-3, pUR, pEX, pMR100, pCR4, pBAD24, pUC19; bacteriophages, such as M1 3 phage and k phage. Of course, such expression vectors may only be suitable for particular host cells. One of ordinary skill in the art, however, can readily determine through routine experimentation whether any particular expression vector is suited for any given host cell. For example, the expression vector can be introduced into the host cell, which is then monitored for viability and expression of the sequences contained in the vector. In addition, reference may be made to the relevant texts and literature, which describe expression vectors and their suitability to any particular host cell.

Host Cells of the Invention

The invention herein relates to genetically modified host cells having vectors containing isolated polynucleotides containing a first polynucleotide sequence encoding SEQ ID NO: 1 linked within one open reading frame to a second polynucleotide sequence.

“Host cell” and “host microorganism” are used interchangeably herein to refer to a living biological cell that can be transformed via insertion of recombinant DNA or RNA. Such recombinant DNA or RNA can be in an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus. The host cells of the present invention may be genetically modified in that isolated polynucleotides have been introduced into the host cells, and as such the genetically modified host cells do not occur in nature. The suitable host cell is one capable of expressing at least one nucleic acid construct or vector encoding at least one polypeptide.

Any prokaryotic or eukaryotic host cell may be used in the present invention so long as it remains viable after being transformed with a sequence of nucleic acids. Preferably, the host cell is not adversely affected by the transduction of the necessary nucleic acid sequences, the subsequent expression of the polypeptides, or the resulting intermediates. In certain embodiments, the host cell is bacterial, and in some embodiments, the bacteria are E. coli. In other embodiments, the bacteria are cyanobacteria. Additional examples of bacterial host cells include, without limitation, those species assigned to the Escherichia, Enterobacter, Azotobacter, Erwinia, Bacillus, Pseudomonas, Klebsiella, Proteus, Salmonella, Serratia, Shigella, Rhizobia, Vitreoscilla, Synechococcus, Synechocystis, and Paracoccus taxonomical classes. Suitable eukaryotic cells include, but are not limited to, fungal, plant, insect or mammalian cells. Suitable fungal cells are yeast cells, such as yeast cells of the Saccharomyces genus. In some embodiments the eukaryotic cell is an algae, e.g., Chlamydomonas reinhardtii, Scenedesmus obliquus, Chlorella vulgaris, or Dunaliella salina.

In some embodiments, the host cell is one that contains a cell wall, such as plant cells, bacteria, fungal cells, algal cells, and some archaea.

Polypeptides of the Invention

The invention herein relates to recombinant polypeptides containing the amino acid sequences encoded by the polynucleotides of the invention. The invention herein further relates to an isolated polypeptide containing SEQ ID NO: 1 conjugated to an atom or a molecule. The atom or molecule may be, for example, a fluorophore, a radionuclide, a toxin, a polymer, a fragrance particle, a small molecule, a polypeptide, or a peptide.

In some embodiments, the isolated polypeptide containing SEQ ID NO: 1 may be conjugated to a detectable label. Examples of detectable labels include radioisotopes (radionuclides) such as ³H, ¹¹C, ¹⁴C, ¹⁸F, ³²P, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹³³Xe, ¹⁷⁷Lu, ²¹¹At, or ²¹³Bi, fluorescent labels such as rare earth chelates (europium chelates), fluorescein types including FITC, 5-carboxyfluorescein, 6-carboxy fluorescein; rhodamine types including TAMRA; dansyl; Lissamine; cyanines; phycoerythrins; Texas Red; and analogs thereof, and enzymatic labels such as luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRP), alkaline phosphatase (AP), β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like.

The isolated polypeptides may be prepared by several routes, employing organic chemistry reactions, conditions, and reagents known to those skilled in the art.

Methods of Increasing the Ability of a Recombinant Protein to Bind to a Carbohydrate and of Identifying a Protein Having an Ability to Bind to a Carbohydrate

The invention herein relates to methods of increasing the ability of a recombinant protein to bind to a carbohydrate. The methods include linking an isolated polynucleotide encoding SEQ ID NO: 1 to an isolated polynucleotide encoding a polypeptide, a peptide, or a protein tag. The linked polynucleotides encode a recombinant protein that has an increased ability to bind to a carbohydrate compared to the polypeptide, peptide, or protein tag on its own.

Linking Polynucleotides

The isolated polynucleotides of the invention are prepared by any suitable method known to those of ordinary skill in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteuci et al. (1980) Tet. Lett. 521:719; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired nucleic acid sequence from the gel via techniques known to those of ordinary skill in the art, such as utilization of polymerase chain reactions (PCR; e.g., U.S. Pat. No. 4,683,195).

Each polynucleotide of the invention can be incorporated into an expression vector. “Expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Preferred expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

Incorporation of the individual polynucleotides may be accomplished through known methods that include, for example, the use of restriction enzymes (such as BamHI, EcoRI, Hhal, Xhol, Xmal, and so forth) to cleave specific sites in the expression vector, e.g., plasmid. The restriction enzyme produces single stranded ends that may be annealed to a polynucleotide having, or synthesized to have, a terminus with a sequence complementary to the ends of the cleaved expression vector. Annealing is performed using an appropriate enzyme, e.g., DNA ligase. As will be appreciated by those of ordinary skill in the art, both the expression vector and the desired polynucleotide are often cleaved with the same restriction enzyme, thereby assuring that the ends of the expression vector and the ends of the polynucleotide are complementary to each other. In addition, DNA linkers maybe used to facilitate linking of nucleic acids sequences into an expression vector.

A series of individual polynucleotides can also be combined by utilizing methods that are known to those having ordinary skill in the art (e.g., U.S. Pat. No. 4,683,195). For example, each of the desired polynucleotides can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other Extension of this overlap by DNA polymerase produces a molecule in which the original sequences are “spliced” together. In this way, a series of individual polynucleotides may be “spliced” together and subsequently transduced into a host cell simultaneously. Thus, expression of each of the plurality of polynucleotides is effected.

Individual polynucleotides, or “spliced” polynucleotides, are then incorporated into an expression vector. The invention is not limited with respect to the process by which the polynucleotide is incorporated into the expression vector. Those of ordinary skill in the art are familiar with the necessary steps for incorporating a polynucleotide into an expression vector.

Expressing Linked Polynucleotides in a Host Cell

The methods of the invention may include expressing the linked polynucleotides in a host cell. Expression of the polynucleotides preferably results in the production of a recombinant protein.

The expression vectors of the invention must be introduced or transferred into the host cell. Such methods for transferring the expression vectors into host cells are well known to those of ordinary skill in the art. For example, one method for transforming E. coli with an expression vector involves a calcium chloride treatment wherein the expression vector is introduced via a calcium precipitate. Other salts, e.g., calcium phosphate, may also be used following a similar procedure. In addition, electroporation (i.e., the application of current to increase the permeability of cells to nucleic acid sequences) may be used to transfect the host cell. Also, microinjection of the nucleic acid sequencers) provides the ability to transfect host cells. Other means, such as lipid complexes, liposomes, and dendrimers, may also be employed. Those of ordinary skill in the art can transfect a host cell with a desired sequence using these or other methods.

In certain embodiments, the linked polynucleotides are expressed in plant host cells. There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276). The principle methods of causing stable integration of exogenous DNA into plant genomic DNA include two main approaches:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. The Agrobacterium system is especially viable in the creation of transgenic dicotyledonous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

In certain embodiments in which plant host cells are used, viruses may be used for introducing the polynucleotides of the invention into host cells. Viruses that have been shown to be useful for the transformation of plant hosts include CaV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral foreign genes in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, the constructions can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression of non-viral foreign genes in plants is demonstrated by the above references as well as in U.S. Pat. No. 5,316,931.

The vector used in the methods of the invention may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host, is integrated into the genome and replicated together with the chromosome(s) into which it has been integrated. Furthermore, a single vector or plasmid or two or more vectors or plasmids which together contain the total DNA to be introduced into the genome of the host, or a transposon may be used.

The vectors preferably contain one or more selectable markers which permit easy selection of transformed hosts. A selectable marker is a gene the product of which provides, for example, biocide or viral resistance, resistance to heavy metals, prototrophy to auxotrophs, and the like. Selection of bacterial cells may be based upon antimicrobial resistance that has been conferred by genes such as the amp, gpt, neo, and hyg genes.

Suitable markers for yeast hosts are, for example, ADE2, HIS3, LEU2, LYS2, MET3, TRP1, and URA3. Selectable markers for use in a filamentous fungal host include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in Aspergillus are the amdS and pyrG genes of Aspergillus nidulans or Aspergillus oryzae and the bar gene of Streptomyces hygroscopicus. Preferred for use in Trichoderma are bar and amdS. A general review of suitable markers for the members of the grass family is found in Wilmink and Dons, Plant Mol. Biol. Reptr. (1993) 11:165-185.

The vectors preferably contain an element(s) that permits integration of the vector into the host's genome or autonomous replication of the vector in the cell independent of the genome.

For integration into the host genome, the vector may rely on the gene's sequence or any other element of the vector for integration of the vector into the genome by homologous or nonhomologous recombination. Alternatively, the vector may contain additional nucleotide sequences for directing integration by homologous recombination into the genome of the host. The additional nucleotide sequences enable the vector to be integrated into the host genome at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which are highly homologous with the corresponding target sequence to enhance the probability of homologous recombination. The integrational elements may be any sequence that is homologous with the target sequence in the genome of the host. Furthermore, the integrational elements may be non-encoding or encoding nucleotide sequences. On the other hand, the vector may be integrated into the genome of the host by non-homologous recombination.

In certain embodiments in which plant host cells are used, sequences suitable for permitting integration of the heterologous sequence into the plant genome are recommended. These might include transposon sequences and the like for homologous recombination as well as Ti sequences which permit random insertion of a heterologous expression cassette into a plant genome.

For autonomous replication, the vector may further comprise an origin of replication enabling the vector to replicate autonomously in the host in question. The origin of replication may be any plasmid replicator mediating autonomous replication which functions in a cell. The term “origin of replication” or “plasmid replicator” is defined herein as a sequence that enables a plasmid or vector to replicate in vivo. Examples of origins of replication for use in a yeast host are the 2 micron origin of replication, ARS1, ARS4, the combination of ARS1 and CEN3, and the combination of ARS4 and CEN6. Examples of origins of replication useful in a filamentous fungal cell are AMA1 and ANS 1 (Gems et al., 1991, Gene 98: 61-67; Cullen et al., 1987, Nucleic Acids Research 15: 9163-9175; WO 00/24883). Isolation of the AMA1 gene and construction of plasmids or vectors containing the gene can be accomplished according to the methods disclosed in WO

More than one copy of a gene may be inserted into the host to increase production of the gene product. An increase in the copy number of the gene can be obtained by integrating at least one additional copy of the gene into the host genome or by including an amplifiable selectable marker gene with the nucleotide sequence where cells containing amplified copies of the selectable marker gene, and thereby additional copies of the gene, can be selected for by cultivating the cells in the presence of the appropriate selectable agent.

The host cell is transformed with at least one expression vector. When only a single expression vector is used (without the addition of an intermediate), the vector will contain all of the nucleic acid sequences necessary.

Once the host cell has been transformed with the expression vector, the host cell is allowed to grow. Methods of the invention may include culturing the host cell such that recombinant nucleic acids in the cell are expressed. For microbial hosts, this process entails culturing the cells in a suitable medium. Typically cells are grown at 35° C. in appropriate media. Preferred growth media in the present invention include, for example, common commercially prepared media such as Luria Bertani (LB) broth, Sabouraud Dextrose (SD) broth or Yeast medium (YM) broth. Other defined or synthetic growth media may also be used and the appropriate medium for growth of the particular host cell will be known by someone skilled in the art of microbiology or fermentation science. Temperature ranges and other conditions suitable for growth are known in the art (see, e.g. Bailey and Ollis, Biochemical Engineering Fundamentals, McGraw-Hill Book Company, NY, 1986.)

Isolating Carbohydrate-Bound Recombinant Proteins

The methods of the invention may include isolating recombinant proteins containing SEQ ID NO: 1 linked to a peptide, polypeptide, or protein tag that are bound to a carbohydrate. In some embodiments, carbohydrate-bound recombinant proteins may be isolated by allowing them to bind to the cell wall of a host cell, and subsequently isolating the cell wall of the host cell. In other embodiments, a recombinant protein may be isolated by allowing it to bind to a carbohydrate matrix. In further embodiments, a carbohydrate-bound recombinant protein may be isolated by other means of affinity purification and chromatography known to those of skill in the art.

Testing Recombinant Proteins for Activity on Carbohydrate Substrates

The methods of the invention may include the step of testing the recombinant protein for its ability to act on a carbohydrate. Testing may include assaying for the degradation, modification, or creation of glycosidic bonds on a carbohydrate substrate. Examples of assays that may be used are enzymatic activity assays, qualitative binding assays, isothermal titration calorimetric analysis of binding, and other assays known to one of skill in the art. Assays may test for glycoside hydrolase activity, glycosyl transferase activity, carbohydrate esterase activity, polysaccharide lyase activity, or carbohydrate binding activity. Carbohydrates substrates include, for example, insoluble carbohydrates and carbohydrates containing hemicellulose.

Detecting Carbohydrate-Bound Recombinant Proteins

Methods of the invention may include detecting recombinant proteins containing SEQ ID NO: 1 linked to a peptide, polypeptide, or protein tag that are bound to a carbohydrate by incubating the carbohydrate-bound recombinant protein with an antibody specific to the polypeptide, peptide, or protein tag that is linked to SEQ ID NO: 1. In certain embodiments, the antibodies may be linked to reporter enzymes such as chromogenic enzymes to allow for detection of the recombinant proteins. In other embodiments, the antibodies that are bound to the recombinant proteins may be detected by secondary antibodies linked to fluorophores or to reporter enzymes. Any other antibody detection system known to one of skill in the art may also be used.

Identifying Proteins Having an Ability to Bind to a Carbohydrate

The invention herein further relates to methods of identifying proteins having an ability to bind to a carbohydrate. The steps of the method include providing a labeled isolated polynucleotide that encodes SEQ ID NO: 1, allowing the labeled polynucleotide to hybridize to a homologous sequence in a nucleotide library, and isolating the sequence bound by the labeled polynucleotide. The sequence may encode a protein having an ability to bind to a carbohydrate.

The isolated polynucleotide may be labeled with radioactive isotopes, enzymes (especially a peroxidase, an alkaline phosphatase, or an enzyme capable of hydrolyzing a chromogenic, fluorigenic or luminescent substrate), chromophoric chemical compounds, chromogenic, fluorigenic or luminescent compounds, nucleotide base analogues, and ligands such as biotin. Hybridization is understood to mean the process during which, under appropriate conditions, two nucleotide sequences, having sufficiently complementary sequences, are capable of forming a double strand with stable and specific hydrogen bonds. The hybridization conditions are determined by the stringency of the operating conditions. The higher the stringency, the more specific the hybridization will be. The stringency is defined especially according to the base composition of a probe/target duplex, as well as by the degree of mismatch between two nucleic acids. The stringency may also depend on the reaction parameters, such as the concentration and the type of ionic species present in the hybridization solution, the nature and the concentration of the denaturing agents and/or the hybridization temperature.

The stringency of the conditions under which a hybridization reaction should be carried out will depend mainly on the nucleotides used. All these parameters are well known and the appropriate conditions can be determined by persons skilled in the art. In general, depending on the length of the probes used, the temperature for the hybridization reaction is between about 20 and 65° C., in particular between 35 and 65° C. in a saline solution at a concentration of about 0.8 to 1M. Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

The nucleotide library used in the method may be a cDNA library or a genomic library. A library contains a collection of cloned nucleotide molecules each inserted into a cloning vector. A genomic library consists of fragments of the entire genome, whereas a cDNA library consists of copies of all of the messenger RNAs produced by a specific cell type.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

The invention having been described, the following examples are offered to illustrate the subject invention by way of illustration, not by way of limitation.

### EXAMPLES

### Example 1—Domain Organization of FSUAxe6B and Proteins Harboring Fibrobacter succinogenes-Specific Paralogous Domain-1 (FPd-1)

Through analysis of the genome sequence of F. succinogenes S85, a gene cluster was identified that encodes more than 10 hemicellulose-targeting enzymes. Most of the enzymes in the cluster are modular polypeptides, a common feature in many carbohydrate active enzymes. Kam and co-workers (16) previously identified 2 acetyl xylan esterases (Axe6A and Axe6B) in this cluster and predicted that each gene encoded a polypeptide composed of two domains: an esterase catalytic domain and a family 6 carbohydrate-binding module. Whereas Axe6A was fairly well characterized, difficulties in expression of recombinant Axe6B restricted its characterization (16).

Based on the amino acid sequence identity, carbohydrate esterases (CEs) have been classified into 16 families (CE1-CE16) according to the CAZy (Carbohydrate Active Enzyme) database (available at www.cazy.org). A domain of FSUAxe6B, from amino acid position 30 to position 329, showed 46% identity to the polypeptide sequence of F. succinogenes acetylxylan esterase Axe6A, a member of carbohydrate esterase family 6 (CE6). Therefore, FSUAxe6B was predicted to be a member of the CE6 family. Further analysis suggested that FSUAxe6B is a modular protein composed of the CE6 domain, a family 6 carbohydrate-binding module (CBM6), and a C-terminal domain of unknown function. Acetylxylan esterase is one of a set of enzymes that is required for xylan deconstruction. This enzyme cleaves ester bonds that link acetyl side groups to the 13-1,4-linked xylopyranoside backbone of xylan, and members of CBM6 are known to bind to a variety of substrates (4, 13, 14, 28, 36).

Although the likelihood that the CBM6 may include the region demarcated as harboring the unknown function was initially considered, this scenario would make the FSUAxe6B CBM unusually long. Therefore, the GenBank database was searched to determine whether the sequence of unknown function occurs in other polypeptides, especially CBM6 proteins, already reported from other organisms. Interestingly, the results yielded no polypeptide with obvious similarity in amino acid sequence to this region. On the other hand, a search of the genome database of F. succinogenes S85 suggested that 23 other proteins harbor amino acid sequences that are similar to this C-terminally located domain of FSUAxe6B. These sequences were designated Fibrobacter succinogenes-specific paralogous domain-1 (FPd-1).

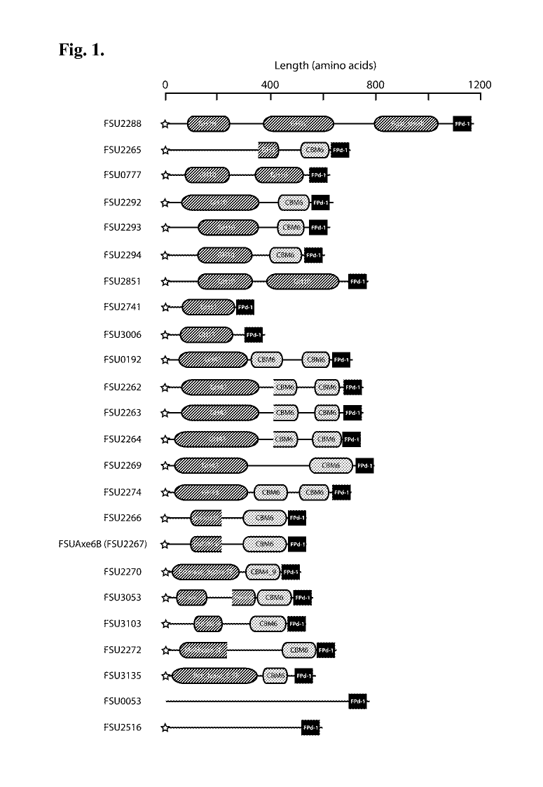

FIG. 1 shows the domain organizations of proteins harboring FPd-1 sequences. Most of these proteins, except for FSU0053, included signal peptides for secretion, suggesting that they function either extracellularly or in the periplasmic space. Among these 24 proteins, 15 proteins harbored glycosyl hydrolase (GH) family domains, which included a GH family 2 protein (GH2) (FSU2288), a GH3 protein (FSU2265), five GH10 proteins (FSU0777, FSU2292, FSU2293, FSU2294, and FSU2851), two GH11 proteins (FSU2741, and FSU3006), and six GH43 proteins (FSU0192, FSU2262, FSU2263, FSU2264, FSU2269, and FSU2274). Additionally, five of the proteins (FSU2266, FSU2267 (Axe6B), FSU2270, FSU3053, and FSU3103) were putative esterases. Whereas one of the gene products was predicted to be a melibiase (FSU2272), another was similar to a pectate lyase (FSU3135). However, no conserved domains were identified in FSU0053 and FSU2516, although the BLAST search suggested that these proteins may contain pectate lyase activity.

It was noted that each of the proteins belonged to protein families that are related to hemicellulose or pectin metabolism. Seventeen of the proteins (FSU0192, FSU2262, FSU2263, FSU2264, FSU2265, FSU2266, FSU2267, FSU2269, FSU2270, FSU2272, FSU2274, FSU2292, FSU2293, FSU2294, FSU3053, FSU3103, and FSU3135) harbored, in addition to FPd-1, either single or double CBM domains, further suggesting that the FPd-1 sequence plays a role in the recognition or catalysis of certain carbohydrates. The FPd-1 domains were consistently located at the C-terminal end of these proteins, and CBMs, when present, were located N-terminal to the FPd-1 domains. We also noted that seven proteins (FSU0053, FSU0777, FSU2288, FSU2516, FSU2741, FSU2851, and FSU3006) that have the FPd-1 domains did not have identifiable CBM sequences, suggesting that the FPd-1 is likely functionally independent of the CBM.

Methods

The genome sequence of F. succinogenes S85 was determined by the North American Consortium for Genomics of Fibrolytic Ruminal Bacteria in collaboration with the Institute for Genomic Research (TIGR) (FibRumba database available at www.jcvi.org/rumenomics). Functional domain search was performed to determine the protein family and domain organization using the Pfam search server (available at www.sanger.ac.uk/Software/Pfam) and NCBI BLAST server (available at www.ncbi.nlm.nih.gov/BLAST). Prediction of lipoproteins and signal peptides was performed by using LipoP 1.0 server (available at www.cbs.dtu.dk/services/LipoP).

### Example 2—FSUAxe6B Truncational Derivatives

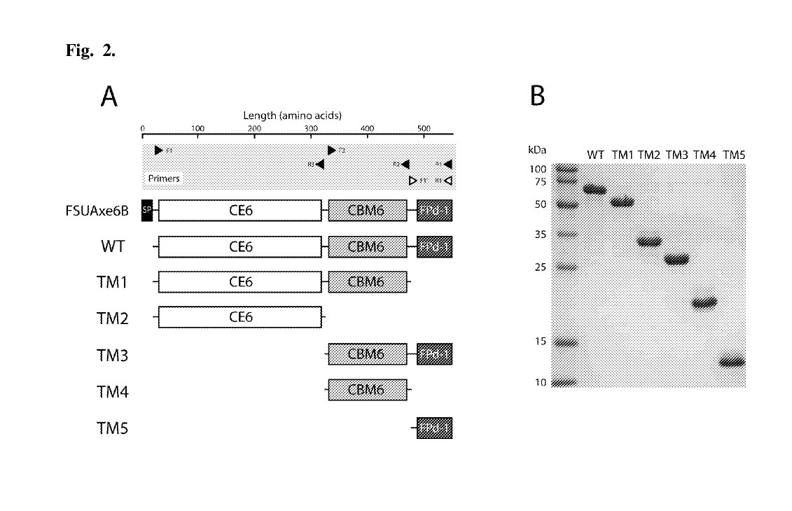

To delineate and investigate the modules present in FSUAxe6B for functional role assignments, a gene truncation strategy was adopted. To create the truncated proteins, the glycines in loop regions were selected as the terminal amino acids of our constructs. Based on the secondary structure analysis, five truncational derivatives of the polypeptide, as shown in FIG. 2A, were made. The construct TM1 (CE6+CBM6) was designed to investigate the contribution of FPd-1 to the wild-type (WT) protein in terms of its catalytic (esterase) and carbohydrate binding activities. Likewise, TM2 (CE6) was constructed for identifying the role of the putative CBM6 on the two potential functions of the protein. TM3 (CBM6+FPd-1), TM4 (CBM6), and TM5 (FPd-1) were constructed for direct determination of the functions of the putative CBM6 and FPd-1 domains. All truncated derivatives of FSUAxe6B were successfully expressed in E. coli as soluble proteins and purified to near homogeneity (FIG. 2B).

Methods

Strains, media, and growth conditions—Fibrobacter succinogenes subsp. succinogenes S85 was obtained from the culture collection at the Department of Animal Sciences, University of Illinois at Urbana-Champaign. F. succinogenes S85 was grown in a synthetic medium (32) under anaerobic conditions. Escherichia coli JM109 and E. coli BL21 (DE3) CodonPlus RIPL competent cells were purchased from Stratagene (La Jolla, Calif.). Gene manipulation and plasmid construction were performed in E. coli JM109. E. coli BL21 (DE3) CodonPlus RIPL was used for gene expression. The E. coli cells were grown aerobically at 37° C. in Luria-Bertani (LB) medium supplemented with appropriate antibiotics.

Gene cloning, expression, and protein purification—F. succinogenes S85 was grown for 2 days, cells were harvested, and the genomic DNA was extracted using DNeasy Tissue kit (QIAGEN, Hilden, Germany). The genes of wild-type FSUAxe6B (WT) and its truncational mutant proteins (TM1, TM2, TM3, and TM4) were amplified from the genomic DNA by PCR using Prime STAR HS DNA polymerase (Takara Bio, Otsu, Japan). The forward and reverse primers used for the PCR were engineered to incorporate NdeI and XhoI restriction sites, respectively. The primer pairs used for amplifying the wild-type protein and its truncated derivative TM1, TM2, TM3, and TM4 were F1/R1, F1/R2, F1/R3, F2/R1, and F2/R2, respectively (Table 1 and FIG. 2A). The amplified fragments were cloned into pGEM-T vector (Promega, Madison, Wis.) and subcloned into a modified pET-28a expression vector (Novagen, San Diego, Calif.) that was engineered by replacing the kanamycin resistance gene with that for ampicillin resistance (3). For the construction of the TM5 expression vector, the EK/LIC cloning kit was utilized (Novagen). The TM5 gene was amplified from the genomic DNA with the primers, F1′ and R1′ (Table 1 and FIG. 2A). Both ends of the amplified gene fragment were digested, in the presence of dATP, with the 3′ to 5′ exonuclease activity of T4 DNA polymerase. The resultant fragment was annealed to the pET-46 EK/LIC vector. The gene expression vectors for FSUAxe6B or its truncated derivatives were introduced individually into E. coli BL21 (DE3) CodonPlus RIPL competent cells, and grown in 10 ml of LB medium with ampicillin (100 μg/ml) and chloramphenicol (50 μg /ml) at 37° C. overnight. Each culture was transferred to a fresh LB medium (1 L) with the same antibiotics, and grown until the optical density at 600 nm reached approximately 0.4. For each culture, the temperature for culturing was then decreased to 16° C., and isopropyl β-D-thiogalactopyranoside (IPTG) was added at a final concentration of 0.1 mM to the medium to induce production of the target protein. After 14 hrs, cells were harvested by centrifugation (5,000 rpm, 4° C., 15 min), and re-suspended in 50 ml of lysis buffer (50 mM Tris-HCl, pH 7.5, 300mM NaCl, 20 mM Imidazole). Cells were disrupted by using an EmulsiFlex C-3 cell homogenizer (Avestin Inc., Ottawa, Canada), and the lysate was clarified by centrifugation (15,000 rpm, 4° C., 30 min). The supernatant was filtered through a 0.22 μm pore size Durapore membrane (Millipore, Bedford, Mass.). The filtrate was applied to HisTrap FF 5 ml (GE Healthcare, Piscataway, N.J.) column, and unbound proteins were washed with 20 column volumes of lysis buffer. The bound proteins were eluted with elution buffer (50 mM Tris-HCl, pH 7.5, 300 mM NaCl, 250 mM Imidazole) and the buffer was exchanged to 50 mM Tris-HCl, pH 7.5, 300 mM NaCl by use of a desalting column (HiPrep 26/10 Desalting, GE Healthcare). The latter buffer served as the storage buffer. All columns used in the protein purification steps were fitted to an ÄKTAxpress system (GE Healthcare).

### Example 3—Steady State Kinetic Analysis of FSUAxe6B Wild-Type and its Truncational Derivatives

In order to obtain the basic catalytic information of FSUAxe6B, steady state kinetic analysis was performed. Using tetra-acetyl-xylopyranoside as a substrate yielded a typical Michaelis-Menten plot, and a k_{cat} of 15 s-1 and a Kₘ value of 0.08 mM were determined for this substrate (Table 2). The kinetic analysis was carried out for the two truncational mutant proteins, TM1 and TM2, which harbor the CE6 domain. The TM1 protein exhibited k_{cat} of 15 s-1 and Kₘ value of 0.09 mM, resulting in a kcat/Kₘ of 170 s-1 mM-1 (Table 2). Likewise, the kinetic parameters for TM2 protein were 13 s-1 (k_{cat}) and 0.07 mM (Kₘ), resulting in a k_{cat}/Kₘ of 190 s-1 mM-1 (Table 2). These values were quite similar to those of wild-type protein, indicating that the CBM6 domain and FPd-1 domain of FSUAxe6B have no obvious effect on the esterase activity, at least with the substrate used in this experiment. Also, the activity of TM2 delineated the catalytic region of FSUAxe6B.

Methods

Assays were carried out at 37° C. Five microliters of 1 μM enzyme and 20 μl of R2 enzyme solution (containing acetate kinase, pyruvate kinase, and D-lactose dehydrogenase in 100 mM Tris-HCl, pH 7.4, 3 mM MgCl2) were thoroughly mixed. The tetra-acetyl-xylopyranoside was prepared in 290 _{i—}EL of R1 solution (NADH, ATP, phospho-enol-pyruvate, and pyruvate). The concentrations of the ingredients R1 and R2 solutions were pre-determined by the manufacturer (Megazyme). Both solutions were incubated separately at 37° C. for 3 min to allow equilibration, and then mixed to start the reaction. Initial rates were plotted against the tetra-acetyl-xylopyranoside concentrations, and the kinetic parameters were determined by Michaelis-Menten equation utilizing Graph Pad Prism v5.01.

### Example 4—Binding Studies of FSUAxe6B and its Truncational Derivatives

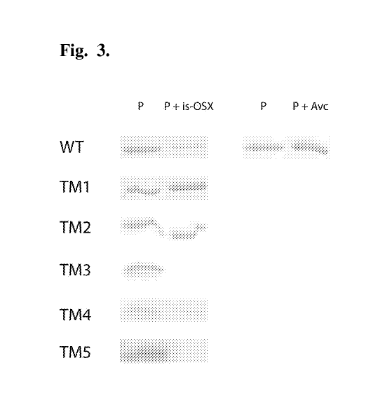

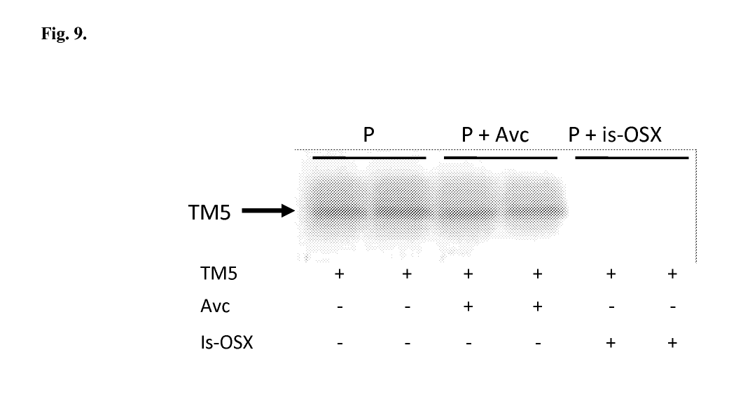

In order to investigate the carbohydrate binding activity of FSUAxe6B, Avicel (crystalline cellulose) and insoluble oat-spelt xylan (is-OSX) were tested as substrates. The WT protein did not show any binding activity to Avicel. However, it showed binding activity for is-OSX (FIG. 3). Furthermore, to identify the location of the FSUAxe6B domains involved in the binding of is-OSX, the truncational derivatives (TM1-TM5) were tested in the binding assays. The qualitative binding assays demonstrated that although TM1 and TM2 have no discernible affinity for is-OSX, TM3, TM4, and TM5 all bound to this substrate (FIG. 3). In addition, TM5 was tested for its ability to bind is-OSX and Avicel (FIG. 9). In these experiments TM5 was capable of binding to is-OSX but not to Avicel. Taken together, these results indicated that the binding activity of FSUAxe6B for is-OSX is located in the TM5 peptide or the region designated as an unknown domain.

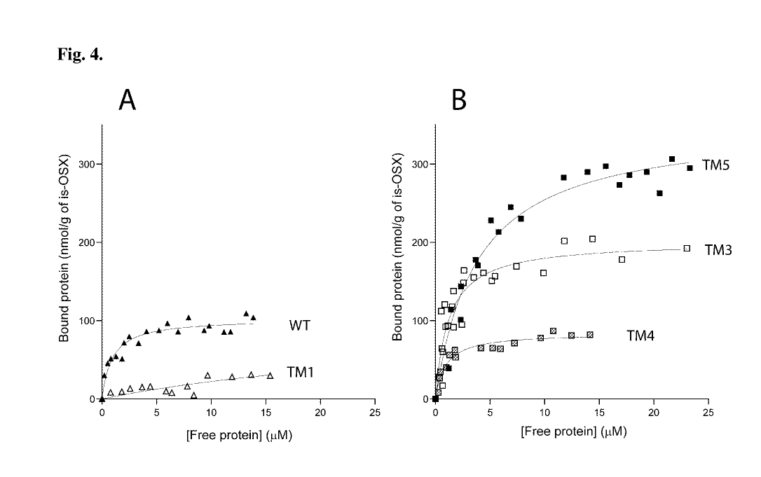

To ascertain these results and to quantify the binding capacity of WT and the truncational mutants (TM1-TM5) for is-OSX, binding isotherms were determined for these proteins. FIG. 4A shows the binding isotherms for the wild-type protein (WT) and the truncated derivative lacking only the FPd-1 (TM1). The truncation of FPd-1 from the wild-type protein led to a dramatic reduction of binding activity for TM1 mutant, suggesting that the FPd-1 domain is key for binding to is-OSX (FIG. 4A), as also observed in the qualitative binding assay (FIG. 3). FIG. 4B shows the binding isotherms for the truncated derivatives that lacked the CE6 catalytic domain. The dissociation constant (K_{d}) of TM5 was 0.26 μM, which is much lower than that of TM4 (K_{d}=1.1 μM). These values showed that the FPd-1 domain (TM5) exhibited much higher binding activity for is-OSX compared to CBM6 domain (TM4), directly indicating that the FPd-1 domain is the true contributor of the binding is-OSX (FIG. 4B and Table 3).

Furthermore, the possibility that TM4 and TM5 domains are one functional domain for binding was investigated. To test this hypothesis, TM3, which is a fusion protein of TM4 and TM5, was tested as well (FIG. 4B). The TM3 protein displayed a K_{d} value of 0.83 μM (Table 3), which is higher than that of TM5, indicating that the binding activity of FSUAxe6B is mainly due to the TM5 domain. Interestingly, the WT exhibited a dissociation constant of 1.1 μM (FIG. 4A and Table 3), which is much higher than that of TM5.

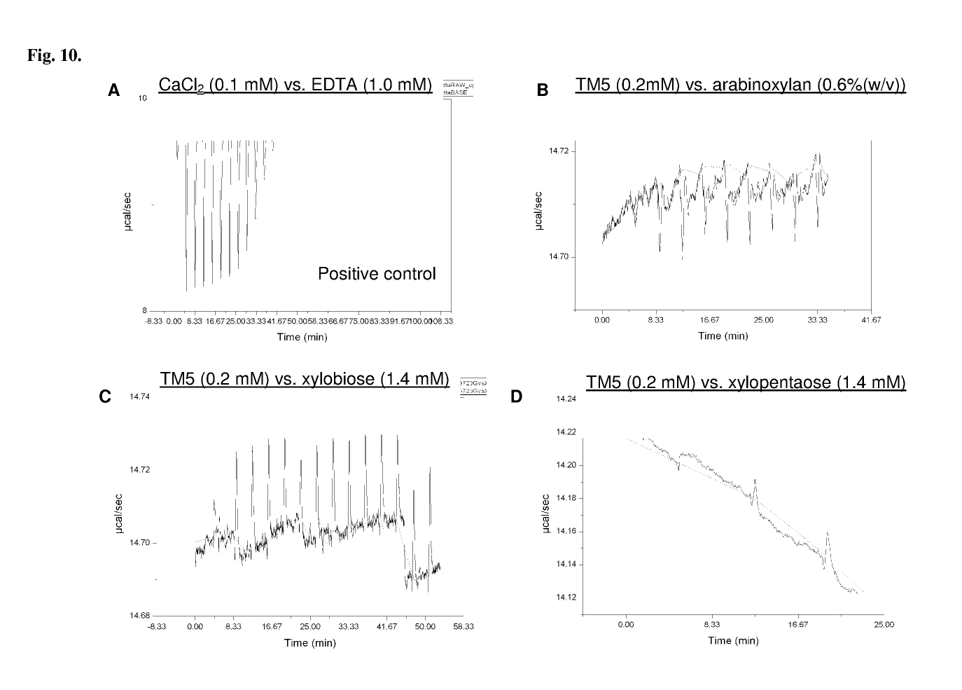

Isothermal titration calorimetric analysis was conducted to determine whether the FPd-1 domain can bind to soluble substrates. TM5 was tested for binding with arabinoxylan, xylobiose, and xylopentaose (FIG. 10). If there is binding affinity between two materials, a binding heat that follows a pattern such as that for CaCl₂ vs. EDTA in the positive control will be observed. However, no significant peaks were observed for TM5 vs. arabinoxylan, xylobiose, or xylopentaose.

Methods

Oat-spelt xylan (OSX) and Avicel PH-101 as ligands were purchased from Sigma-Aldrich (St. Louis, Mo.). Since OSX contains some soluble components, the soluble fraction was excluded as follows. One gram of OSX was stirred in 100 ml of distilled water for 12 h. After centrifugation (4,000×g, 10 min, RT), the precipitate was further washed with 100 ml of distilled water, and centrifuged (4,000×g, 10 min, RT). The insoluble fraction was lyophilized and then ground into small particles in a mortar, producing insoluble oat-spelt xylan (is-OSX). Qualitative binding assessment between proteins and ligands was carried out as follows: One ml of 2 μM proteins in 50 mM Tris-HCl, pH 7.5, containing 300 mM NaCl (Buffer A) was mixed with 20 mg of insoluble polysaccharide. The reaction mixture was gently mixed at 4° C. for 1 hr. Then, the insoluble polysaccharide was precipitated by centrifugation (13,000 rpm, 4° C., 1 min). The supernatants, including unbound protein, were concentrated up to 10 times and 10 μl was loaded and resolved on a 12.5% SDS-PAGE. Blanks (Lane P), for excluding the possibility of precipitation or adsorption of the protein to the tube during reaction, were prepared by incubating the protein without insoluble polysaccharide in the reaction buffer. Depletion binding isotherms were derived for quantitatively assessing the binding capacity of the protein for insoluble polysaccharide. The BCA (bicinchoninic acid) protein assay kit (Thermo scientific, Rockford, Ill.) was used for the quantification of proteins. One ml of various concentrations of proteins in Buffer A was added to 20 mg of is-OSX, and incubated with gentle mixing at 4° C. The supernatant after centrifugation (13,000 rpm, 4° C., 1 min) was used for the quantification of the unbound (free) protein. Total protein was measured after incubating protein without is-OSX under the same conditions. Bound protein was calculated by subtracting the free protein from the total protein.

For isothermal titration calorimetric (ITC) analysis, measurements were performed at 25° C. using a VP-ITC calorimeter (MicroCal, Inc, Northhampton, Mass.) following the manufacturer's recommended procedures. All samples were extensively dialyzed against 50 mM Na₂HPO₄—HCl buffer (pH 7.0), 100 mM NaCl, and all ligands were dissolved in the same buffer. The protein sample (100 μM) was injected with successive 10-μl aliquots of ligand at 300-s intervals.

For the determination of binding constant between protein and ligand, the Michaelis/Langmuir equation was applied. The equation is as follows:

q_{ad}/q=K_{d}*qₘₐₓ/(1+Kₚq)

where q_{ad} is the amount of bound protein (nmol of proteins per g of is-OSX), q is the free protein in buffer (μM), K_{d} is the dissociation constant (μM), and qₘₐₓ is the maximum amount of bound protein to ligand (21). The Graph Pad Prism v5.01 (GraphPad Software, San Diego, Calif.) was utilized for the calculation of the binding parameters.

### Example 5 —Multiple Sequence Alignment of FPd-1 Sequences

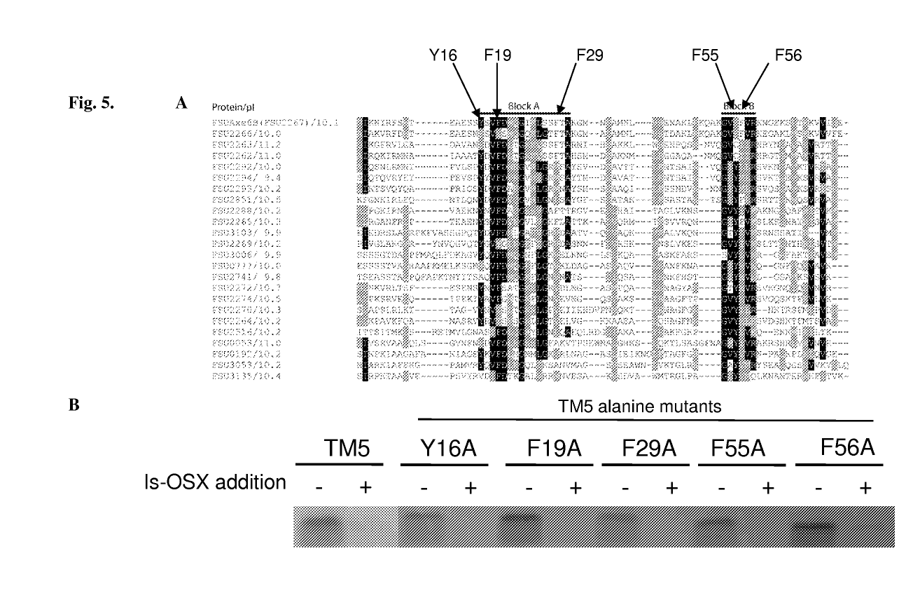

The 24 FPd-1 sequences were aligned using ClustalW (available at www.ebi.ac.uk/clustalw) (FIG. 5A). The alignment revealed two conserved regions (Block A and Block B). Aromatic residues (tryptophan, tyrosine, and phenylalanine) in CBMs generally play a critical role in binding by forming hydrophobic stacking interactions with sugars in the carbohydrate polymer (2). We observed 5 relatively conserved aromatic residues: 1 tyrosine residue and 2 phenylalanine residues in Block A, and 2 phenylalanine residues in Block B (FIG. 5A). To test whether these aromatic residues are critical for binding of FPd-1 to insoluble oat-spelt xylan, single site-directed alanine mutants of TM5 were made and tested for binding to is-OSX (FIG. 5B). 20 mg of insoluble is-OSX was incubated with 1 mL of 10 mM protein, and the supernatant (12.5 mL) was loaded on SDS-PAGE. Lane (−) represents the same amount of protein incubated in the same buffer, but without substrate. The supernatants after incubation of the proteins with is-OSX are shown as (+). No protein in the (+) lane indicates binding to substrate. All TM5 aromatic residue mutants still retained binding affinity for is-OSX.

Another interesting characteristic of the FSUAxe6B protein is the differences of the isoelectric points (pls) of its different modules. The pI of TM2 (esterase domain), TM4 (CBM6), and TM5 (FPd-1) were 5.2, 4.6, and 10.1, respectively. The high pI of TM5 is due to the high proportion of positively charged amino acid residues in its sequence. Consistent with this observation, the other FPd-1 peptides (FIG. 5) also have high pI values ranging from 9.4 for FSU2294 to 11.2 for FSU2263.

### Example 6—Determination of Active Site Residues in FSUAxe6B

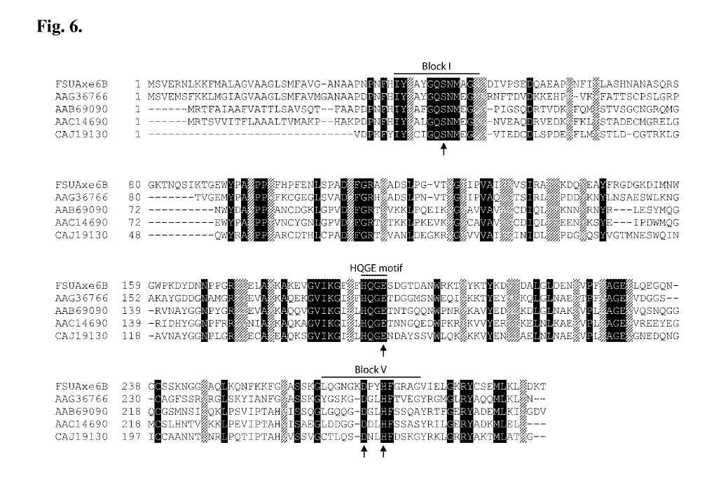

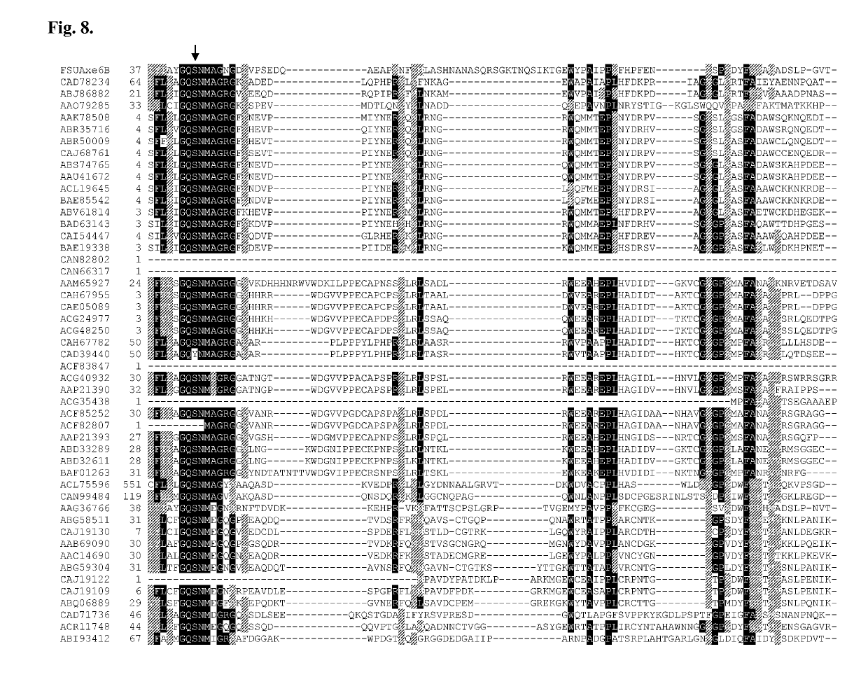

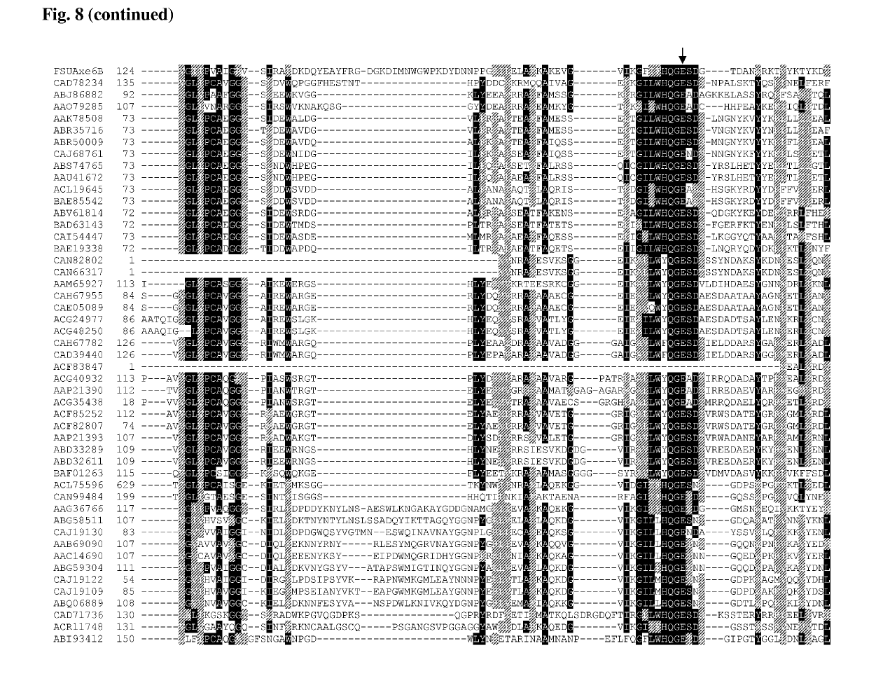

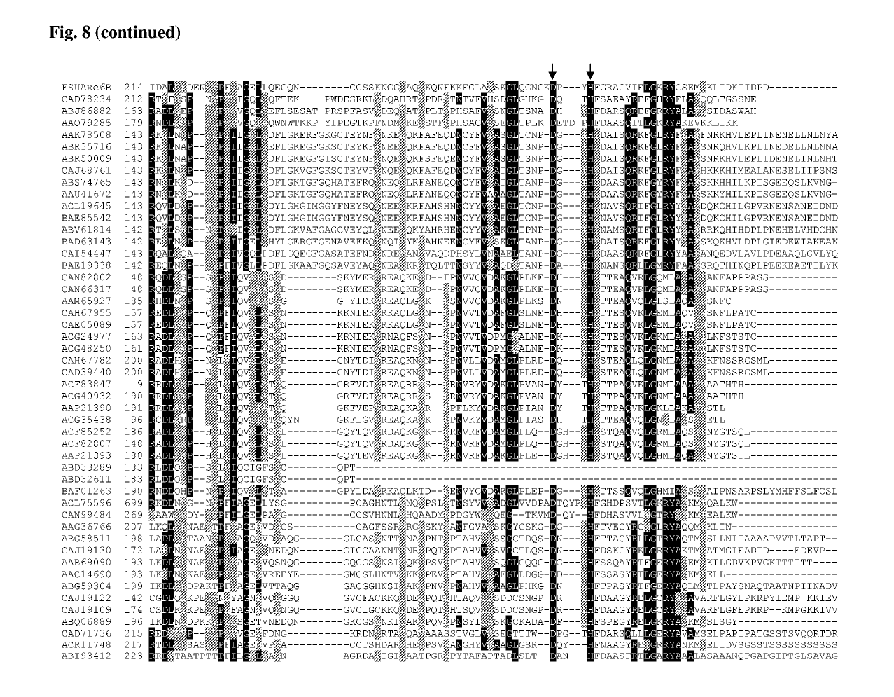

In previous studies on acetylxylan esterases, the deacetylation mechanism of xylan was proposed (11) (12). The catalysis starts with an aspartate, acting as a helper acid, which forms a hydrogen bond with histidine, leading to an increase in the pKa of its imidazole nitrogen. This allows the histidine to become a strong general base, removing a proton from the hydroxyl group of serine. The deprotonated serine serves as a nucleophile and attacks the carbonyl carbon of the acetyl group. This mechanism allows the replacement of aspartate by a glutamate. Indeed, a catalytic triad formed by serine, histidine, and glutamate has been identified for the CE6 family protein R.44 from an uncultured rumen microbe (23). The three residues (Ser14, His231, and Glu152) reside in highly conserved regions in the CE6 family proteins (FIG. 6 and FIG. 8).

The amino acid sequence of FSUAxe6B was compared with that of biochemically characterized CE6 proteins, and the amino acids were found to be completely conserved (FIG. 6) in the F. succinogenes protein. Following the previous study (23), the serine at position 44 of FSUAxe6B (S44), the glutamate at position 194 (E194), and the histidine at position 273 (H273) were mutated to glycine, asparagine, and glutamine, respectively. As expected, the S44G and H273Q mutations abolished detectable activities (Table 4). However, the E194N mutant exhibited detectable catalytic activity.

Thus, a detailed kinetic analysis was conducted, which determined a k_{cat} and Kₘ of 2.8 s-1 and 7 mM, respectively, for E194N. The catalytic efficiency (k_{cat}/Kₘ) of this mutant was 0.40 s-1 mM-1, which is considerably lower compared with that of the wild-type protein (WT) (190 s-1 mM-1). These results indicated that the glutamate at position 194 (E194) is largely contributing to catalysis. We considered the possibility that the replaced asparagine formed a hydrogen bond with histidine by way of its carbonyl group. To ascertain that the E194 is a member of the catalytic residues, it was substituted with alanine (E194A). Surprisingly, E194A also displayed some catalytic activity. The k_{cat} and Kₘ of this mutant were 2.9 s-1 and 0.2 mM, respectively, resulting in a k_{cat}/Kₘ of 14 (Table 4), which is also lower than WT (k_{cat}/Kₘ=190).

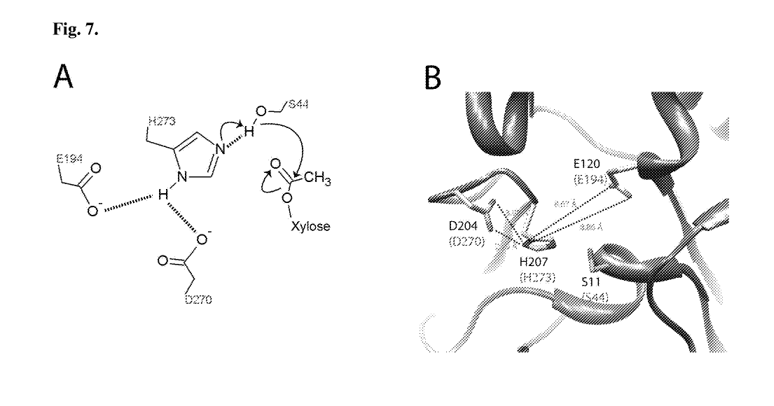

Since mutating E194, located in the vicinity of the catalytic pocket, did not completely abolish catalysis, another residue was sought that could serve as the helper acid in the catalysis. To facilitate the search, FSUAxeB was modeled after the 3-D structure of a Clostridium acetobutylicum putative acetylxylan esterase (PDB number; 1ZMB), the most similar protein structure available in the database. The residues, S44, E194, and H273 in FSUAxe6B are completely conserved in 1ZMB (FIG. 7B). Furthermore, a potential helper acid, an aspartate with 3.39 Å as the mean value (distance) between its ionized group and the nitrogen of the imidazole group in H273, was located. This aspartate is also conserved in FSUAxe6B (D270) (FIG. 7B). Interestingly, the D270A and D270N mutants of FSUAxe6B displayed catalytic activities against tetra-acetyl-xylopyranoside. Thus, the D270A mutant, which showed similar catalytic properties to the D270N mutant, exhibited k_{cat} and Kₘ of 1.8 s-1 and 0.2 mM, respectively. The k_{cat}/Kₘ of D270A is, therefore, 9.0 (Table 4). These kinetic parameters were comparable to those of the E194A mutant. Since no other potential helper acid could be identified, a E194A/D270A double mutant was created. The activity of this mutant was completely abolished (Table 4), suggesting that both E194 and D270 contribute to catalysis, perhaps both residues acting as helper acids.

The circular dichroism (CD) spectra analyses for the WT protein and the mutants were carried out to investigate the structural effects of the amino acid substitutions (Table 5). Among the mutant proteins, D270N and D270A showed similar secondary structural compositions to that of the WT protein. Also, other than the percentage of β-sheets, which was slightly increased, the parameters for the H273Q mutant was not very different from that of the wild-type protein. On the other hand, some increases in a-helix structure were observed for S44G (17% compared with 14% for the wild-type). The percentages of a-helices increased slightly and the percentages of β-sheets decreased slightly for the E194N, E194A and E194A/D270A double mutants compared to the wild type. The corresponding amino acid residues of S44 and E194 in FSUAxe6B are both located in an α-helix structure in the putative acetylxylan esterase from Clostridium acetobutylicum (PDB number; 1ZMB) (FIG. 7B), and this location might be the reason why the proportion of α-helical structures in FSUAxe6B was slightly increased when the residues were mutated. Of much interest are the two mutants E194A and D270A, originally selected as potential helper acids during catalysis. The D270A mutant has almost no detectable structural difference with the wild-type and, although it dramatically decreased esterase activity, it failed to abolish catalytic activity. The E194A mutant, in contrast, exhibited some structural differences compared with the wild-type, but was not very different from the D270A mutant in terms of its catalytic activity. Fascinatingly, however, a double mutant of the two residues E194A/D270A failed to exhibit detectable activity, suggesting that both residues may be critical to catalysis.

Methods

Site-directed mutagenesis—Site-directed mutagenesis was carried out using the QuikChange Multi Site-Directed Mutagenesis Kit (Stratagene), according to the manufacturer's instructions. Primers used in the site-directed mutagenesis study are presented in Table 1.

Bioinformatic analysis—The secondary structure of FSUAxe6B was predicted by using the Advanced Protein Secondary Structure Prediction Server (available at the website of imtech.res.in/raghava/apssp). PDB files were visually analyzed by the UCSF Chimera molecular graphics program (available at www.cgl.ucsf.edu/chimera).

Enzyme assays and steady state kinetics—Acetylxylan esterase activity was assayed using tetra-acetyl-xylopyranoside (Toronto Research Chemicals Inc., Ontario, Canada) for all proteins in this study, and the released of acetic acid was measured using an acetic acid detection kit (Megazyme, Bray, Ireland) following the manufacturer's instructions. The reduction of NADH was monitored continuously at an absorbance of 340 nm using Synergy 2 Microplate reader (BioTek, Winooski, Vt.) using the path-length correction feature. All assays were carried out at 37° C. Five microliters of 1 μM enzyme and 20 μl of R2 enzyme solution (containing acetate kinase, pyruvate kinase, and D-lactose dehydrogenase in 100 mM Tris-HCl, pH 7.4, 3 mM MgCl2) were thoroughly mixed. The tetra-acetyl-xylopyranoside was prepared in 290 μL of R1 solution (NADH, ATP, phospho-enol-pyruvate, and pyruvate). The concentrations of the ingredients R1 and R2 solutions were pre-determined by the manufacturer (Megazyme). Both solutions were incubated separately at 37° C. for 3 min to allow equilibration, and then mixed to start the reaction. For active-site mutants with lower activity, the kinetic parameters were determined at a concentration of 10 μM for E194N protein and 2 μM for E194A, D270N, and D270A proteins, respectively. Initial rates were plotted against the tetra-acetyl-xylopyranoside concentrations, and the kinetic parameters were determined by Michaelis-Menten equation utilizing Graph Pad Prism v5.01.

Circular dichroism (CD) spectra—Determination of CD spectra for the FSUAxe6B wild-type protein (WT) and its site-directed mutant proteins was carried out using a J-815 Circular Dichroism spectropolarimeter (Jasco, Tokyo, Japan). Protein samples were prepared at a concentration of 0.1 mg/ml in 20 mM phosphate (NaH2PO4) buffer (pH 7.5) (17). For the measurements, a quartz cell with a path-length of 0.1 cm was utilized. CD-scans were carried out at 25° C. from 190 nm to 260 nm at a speed of 50 nm/min with a 0.1 nm wavelength pitch, with 5 accumulations. Data files were analyzed on the DICHROWEB on-line server (available at www.cryst.bbk.ac.uk/cdweb/html/home.html) using the CDSSTR algorithm with reference set 4 that is optimized for 190 nm-240 nm (22).

### Example 7

The gram negative rumen bacterium, Fibrobacter succinogenes S85 is estimated to have 104 putative glycoside hydrolases, 4 polysaccharide lyases, and at least 14 carbohydrate esterases from its complete genome information (9). It is clear that this bacterium has well-developed machinery that is devoted to plant cell wall degradation. The abundant carbohydrate active enzymes, along with the modular protein structures, likely endow F. succinogenes S85 with the flexibility to survive on diverse polysaccharides and also to compete in the rumen environment. An example of these versatile proteins is the modular protein FSUAxe6B characterized in this study. The F. succinogenes S85 Axe6A, a protein similar to FSUAxe6B, was shown to possess esterase activity and also to bind to Avicel cellulose, beech-wood xylan and to a lesser extent insoluble oat-spelt xylan (16). A similar characterization for Axe6B was restricted by an inability to express sufficient amounts of recombinant Axe6B. In this study, overexpression, delineation of modules, and biochemical characterization of each module in the FSUAxe6B showed that the polypeptide is composed of a family 6 acetylxylan esterase domain, a carbohydrate-binding module family 6 (CBM6), and surprisingly, an unknown domain, to which we have assigned a function.

Biochemical analysis utilizing the truncational mutants of FSUAxe6B revealed the function of the C-terminal unknown domain as a novel carbohydrate-binding module. In our experiments, the F. succinogenes—specific paralogous domain (FPd-1) clearly bound to insoluble oat-spelt xylan (is-OSX) (FIG. 3 and FIG. 4). Carbohydrate-binding modules (CBMs) are protein folds that recognize specific polysaccharides and are often linked to a catalytic glycoside hydrolase domain through flexible loops (2). Many CBMs have been identified experimentally, and classified into 54 families based on similarity of amino acid sequence (available at www.cazy.org/fam/acc_CBM.html). FPd-1 was proposed to be a novel CBM family because there is no characterized CBM that shares homology with its sequence.

A suggestion has been made to classify CBMs into 3 groups (Type A, Type B, and Type C) based on their structures and functionalities (2). Type A CBMs are defined as surface-binding, and they bind to insoluble cellulose and/or chitin crystals. FPd-1 preferred insoluble xylan, harboring heterogenous amorphous structure (7), to crystalline cellulose (FIG. 3). On the other hand, Type B and Type C CBMs are peptides that are able to bind to soluble polysaccharides using a cleft in their structure. Although we were able to show that the FPd-1 of FSUAxe6B binds to insoluble oat-spelt xylan (is-OSX), our binding experiments with isothermal titration calorimetry suggested that the module does not bind to soluble substrates such as xylobiose, xylopentaose, and soluble arabinoxylan (FIG. 10). Thus, currently FPd-1 cannot be assigned to any of the proposed group of CBMs.

In CBMs, the common binding mechanism is an interaction between aromatic amino acids and the carbohydrates as ligand. The amino acid sequence of FPd-1 in FSUAxe6B showed the presence of a single tyrosine residue, 5 phenylalanine residues and no tryptophan (FIG. 5). Alanine scans for these aromatic residues did not abolish the binding capacity of TM5 for is-OSX (FIG. 5), which suggested that the binding mechanism reported to be mediated by these residues is not critical for FPd-1 or multiple aromatic residues are involved in the interactions with substrate.

Since the initial report on a C-terminal basic domain (BTD) specific to enzymes in F. succinogenes (25), many BTD domains in this strain have been reported (15, 29, 30). To date, the role of the BTD domains remain unknown. From this study's data on FPd-1 s (FIG. 1), all identifiable homologs of this domain are located at the C-terminus of the individual proteins. In addition, they are likely to display basic features (FIG. 5) at neutral pH as generally found in the rumen environment. Thus, similar to the BTDs, the FPd-1s are C-terminally located and also have basic properties. The FPd-1s, therefore, share some common features or properties with the BTDs. However, the amino acid sequences of hitherto reported BTDs are different from those of the FPd-1s identified in this study. In contrast to the unknown function of BTDs, a carbohydrate binding property for a member of the FPd-1s was clearly demonstrated in this study.

It is also of interest that domains that share similar properties with FPd-1s have been observed in proteins from the gram-positive rumen bacterium Ruminococcus albus. The so-called X domains were first reported as C-terminal modules in the cellulose-binding proteins Ce19B and Ce148A through proteomic analysis (6). The domains exhibited a wide binding specificity for ligands and are currently classified as CBM family 37. This CBM family has members reported from only R. albus (37). Recently, a CBM37 domain was demonstrated to be crucial for binding to bacterial cell-surface (8). Similar to the FPd-1 domains in F. succinogenes, the C-terminal ˜100 amino acid sequences (CBM37s) in Ce15G, Ce19C and Ce148A have high pIs as follows; 9.78 (Ce15G), 9.59 (Ce19C) and 9.70 (Ce148A), respectively. The gram-negative F. succinogenes and gram-positive R. albus are two of the major microbes that adhere to and degrade insoluble polysaccharides in the rumen (9). The CBM37s and the FPd-1s may share some common function such as an electrostatic interaction between peptides and cell wall surface.

Many CBM6s have been characterized, and their ligand-specificities have been shown (4, 13, 14, 28, 36). Based on information derived from a previous study (16), the binding sites of biochemically and structurally characterized CBM6s of Cellvibrio mixtus endoglucanase 5A (14, 28) and Clostridium thermocellum xylanase 11A (4) are not conserved in the FSUAxe6B CBM6. In this study, some affinity was detected between the CBM6 domain (TM4) and insoluble oat-spelt xylan. However, the activity was much lower than the FPd-1 domain (TM5). Although the CBM6 of FSUAxe6B is likely to bind to a specific carbohydrate or may exhibit other functional roles for efficient catalysis, in the current study it was difficult to clearly assign a role to it.

The GDS(L) esterase/lipase family possesses a catalytic serine in the conserved motif GDS(L), and it was suggested that this protein family employs a catalytic triad formed by a serine in the Block I consensus sequence, and a histidine and an aspartate in the Block V consensus sequence (1, 5). Although carbohydrate esterase family 6 (CE6) is a member of GDS(L) esterase/lipase family, it was recently demonstrated that the glutamate in the HQGE motif of Block III is the sole catalytic helper acid in R.44 protein (23). In the present study, to determine whether this finding is applicable to FSUAxe6B, a member of the CE6 family, site-directed mutagenesis studies were carried out on the esterase. The serine, as a nucleophile in Block I, and the histidine, as a base to deprotonate the hydroxyl group of the serine in Block V, were identified (FIG. 6, FIG. 7 and Table 4). However, analysis of mutants with a single mutation (E194N, E194A, D270N, and D270A) and a mutant with double mutations E194A/D270A suggested that E194 and D270 may both be important for catalysis, potentially serving as dual helper acids, instead of the single helper acid proposed to function in the deacetylation mechanism described above. The two carboxylates are highly conserved among CE6 family proteins (FIG. 8), and it may be a common catalytic mechanism in this family. Axe6A, with a 61% amino acid sequence similarity to the catalytic domain of Axe6B, exhibited some similarity of kinetic data to Axe6B (Kₘ of 0.08 mM and 0.06 mM for Axe6A and Axe6B, respectively), although the Vₘₐₓ for the two proteins were quite different (16).

### Example 8—Analysis of FPd-1 Domain of FSU2269

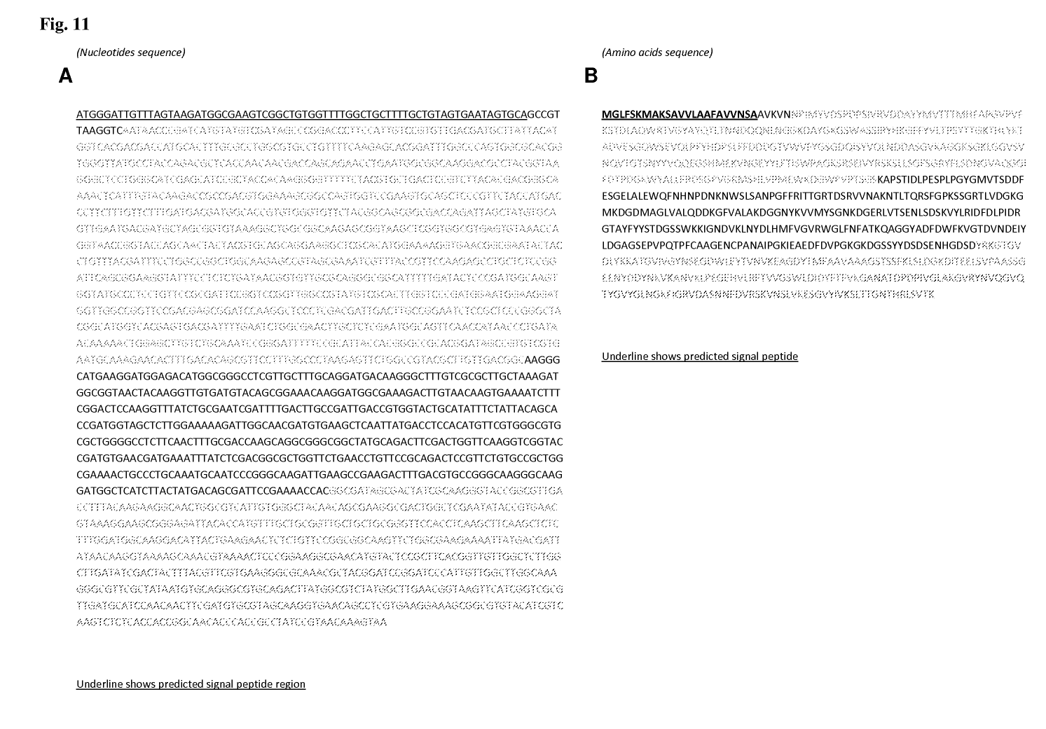

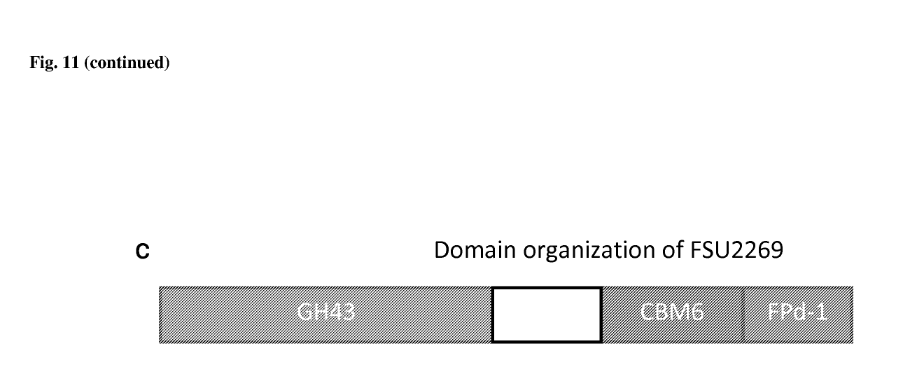

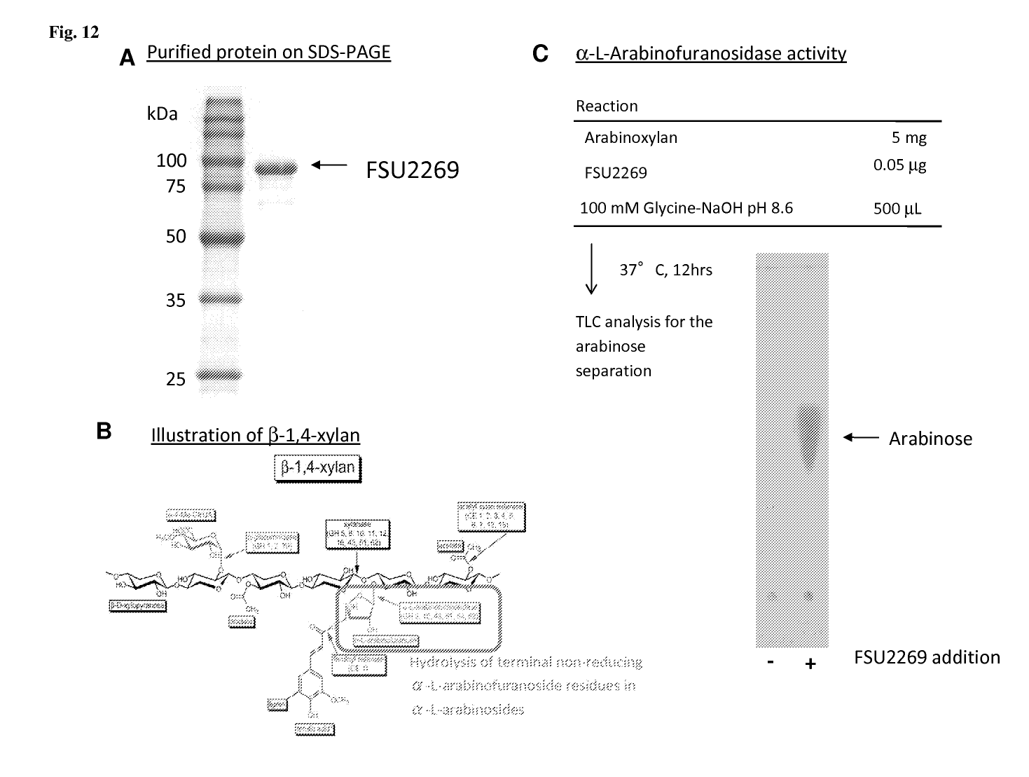

In order to evaluate further the binding characteristics of the FPd-1 domain, the FPd-1 domain of FSU2269, a paralog of FSUAxe6B (FIG. 1), was analyzed. The nucleotide and amino acid sequences of FSU2269 and the predicted domains of the polypeptide are shown in FIG. 11. FSU2269 was expressed and purified. The purified protein on SDS-PAGE with a size of approximately 100 kDa is shown in FIG. 12A. FSU2269 was demonstrated to be an α-L-arabinofuranosidase (FIG. 12). The linkage cleaved by the enzyme is shown in FIG. 12B. Thin layer chromatography showed that in the absence of the enzyme (−), there was no release of product. However, when FSU2269 was added, products (arabinose) were released (FIG. 12C).

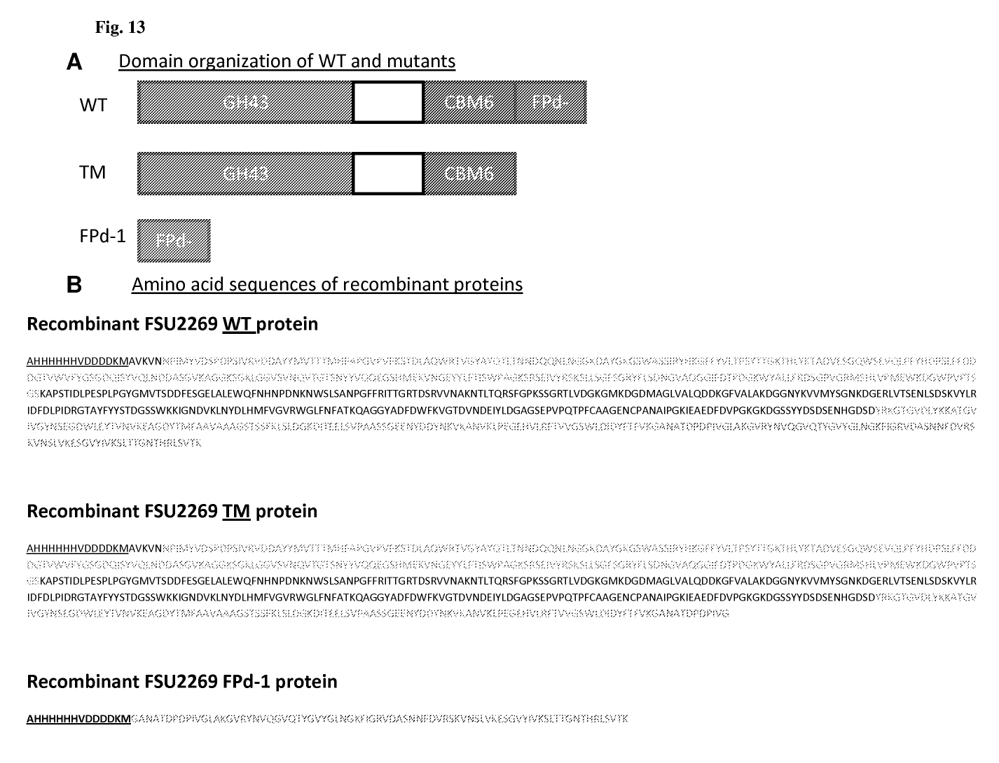

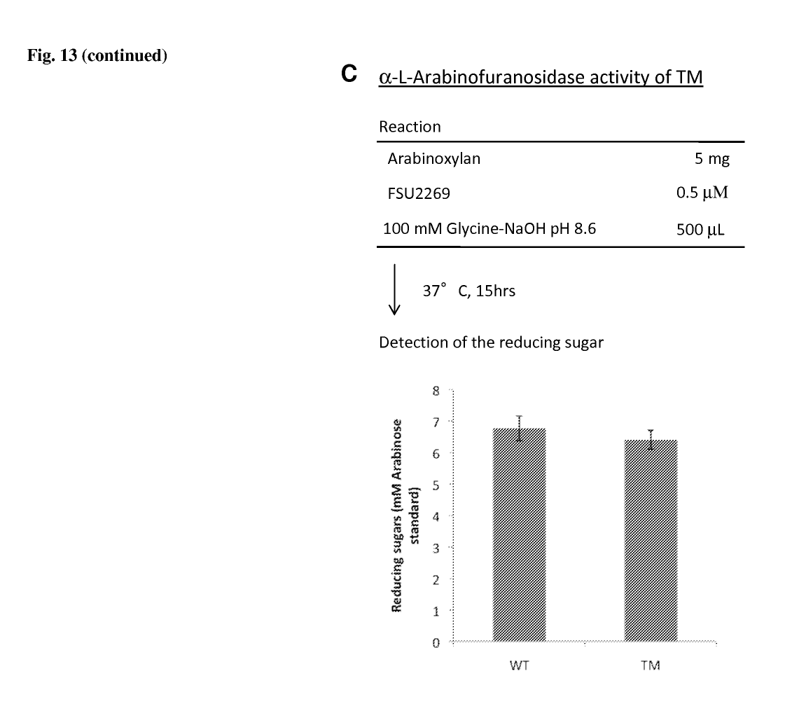

Truncational mutants of FSU2269 were generated (FIG. 13A). Each protein was expressed with an N-terminal 6 Histidine tag from the plasmid (FIG. 13B). The wild type protein (WT) released arabinose from the substrate (arabinoxylan) (FIG. 13C). If FPd-1 is cleaved from the polypeptide, the truncated protein (TM) was still active as an arabinofuranosidase (FIG. 13C).

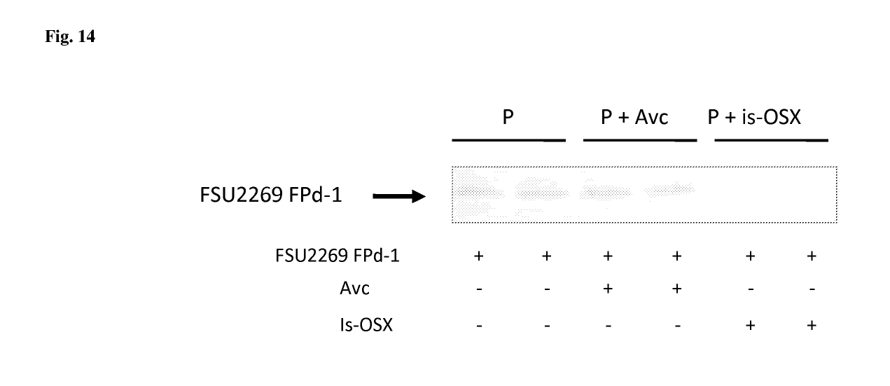

Qualitative binding assays of FSU2269 FPd-1 were performed for Avicel and is-OSX (FIG. 14). Methods were the same as described in Example 4. FSU2269 FPd-1 was capable of binding is-OSX but not Avicel as was also found for the FsuAxe6B FPd-1.

### Example 9—Determination of a Consensus Sequence for FPd-1

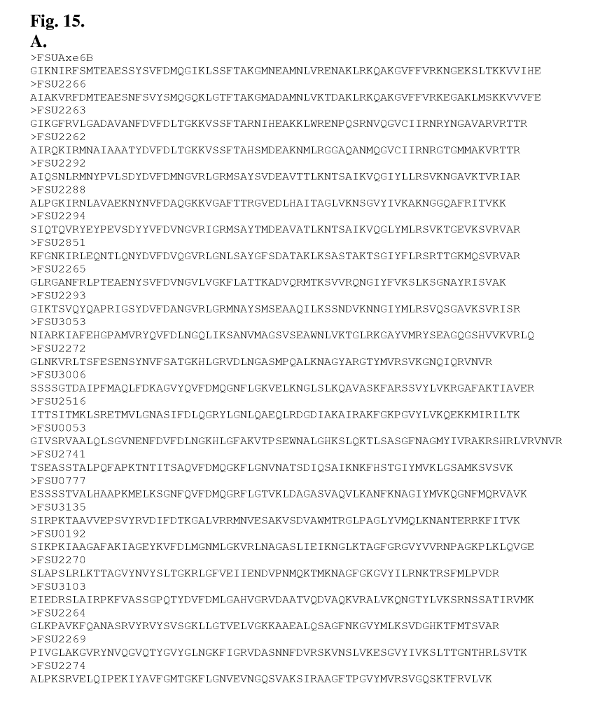

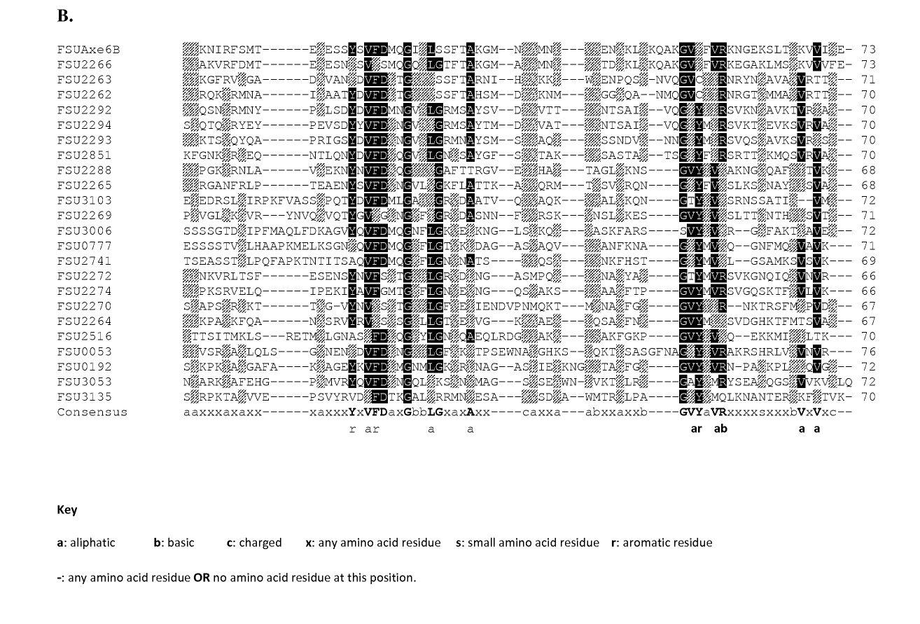

In order to determine a consensus sequence for the FPd-1 domain, an alignment was generated with ClustalW2 (available at www.ebi.ac.uk/Tools/clustalw2/index.html) (FIG. 15). Shading was carried out manually according to the key shown at the bottom of FIG. 15B. Conserved and similar amino acid residues occurring at 50% or more at a single position were shaded black and gray, respectively. The consensus sequence follows the key and where two residues occurred at a single position, the bolded letter represents the conserved residue, which may also be substituted for by the letter below. The key in FIG. 15B indicates the definition of this letter. Thus, the consensus sequence for FPd-1 was determined to be

(SEQ ID NO: 1)
aaxxxaxaxx------xaxxxYxVFDaxGbbLGxaxAxx----caxxa-
--abxxaxxb----GVYaVRxxxxsxxxbVxVxc-.

### Example 10—Analysis of FPd-1 Domains of Additional F. succinogenes Proteins

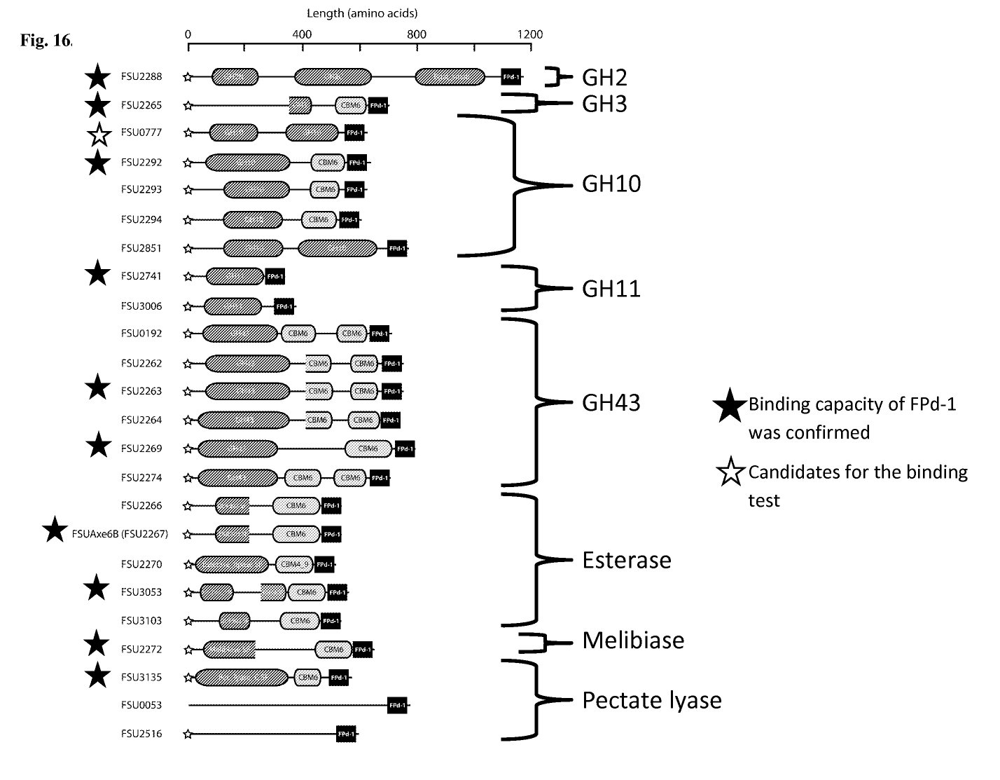

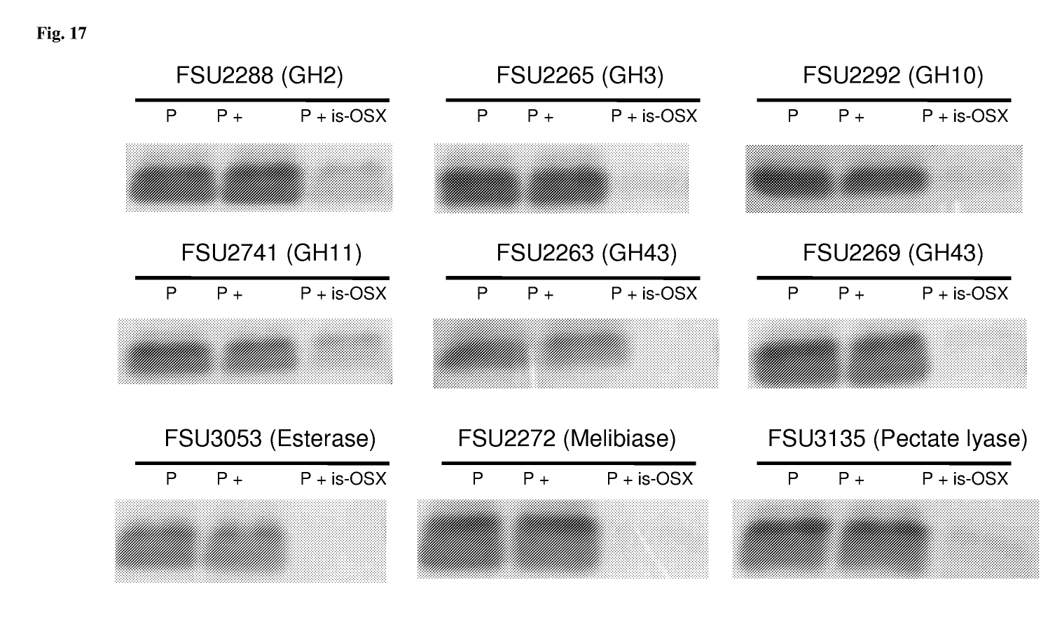

The FPd-1 domains of the F. succinogenes proteins marked with a black star in FIG. 16 were cloned, expressed, and purified. An amount of 20 mg of Avicel PH-101 (Avc) or insoluble oat-spelt xylan (is-OSX) was incubated with 1 mL of 2 μM FPd-1 peptide. After incubation of the proteins with is-OSX or Avc, the supernatants were concentrated up to 10 times, and 10 μL of the resulting solution was loaded on SDS-PAGE as (P+Avc) and (P+is-OSX), respectively. Lane P represents the same amount of protein incubated in the same buffer, but without substrate. All FPd-1 proteins clearly bound to is-OSX, but no significant binding to Avicel PH-101 was observed.

### REFERENCES 1. Akoh, C. C., G. C. Lee, Y. C. Liaw, T. H. Huang, and J. F. Shaw. 2004. GDSL family of serine esterases/lipases. Prog. Lipid Res. 43:534-52.

2. Boraston, A. B., D. N. Bolam, H. J. Gilbert, and G. J. Davies. 2004. Carbohydrate-binding modules: fine-tuning polysaccharide recognition. Biochem. J. 382:769-81.

3. Cann, I. K. O., S. Ishino, M. Yuasa, H. Daiyasu, H. Toh, and Y. Ishino. 2001. Biochemical analysis of replication factor C from the hyperthermophilic archaeon Pyrococcus furiosus. J. Bacteriol. 183:2614-23.

4. Czjzek, M., D. N. Bolam, A. Mosbah, J. Allouch, C. M. G. A. Fontes, L. M. A. Ferreira, 0. Bornet, V. Zamboni, H. Darbon, N. L. Smith, G. W. Black, B. Henrissat, and H. J. Gilbert. 2001. The location of the ligand-binding site of carbohydrate-binding modules that have evolved from a common sequence is not conserved. J. Biol. Chem. 276:48580-7.

5. Dalrymple, B. P., D. H. Cybinski, I. Layton, C. S. McSweeney, G. P. Xue, Y. J. Swadling, and J. B. Lowry. 1997. Three Neocallimastix patriciarum esterases associated with the degradation of complex polysaccharides are members of a new family of hydrolases. Microbiology 143:2605-14.

6. Devillard, E., D. B. Goodheart, S. K. Karnati, E. A. Bayer, R. Lamed, J. Miron, K. E. Nelson, and M. Morrison. 2004. Ruminococcus albus 8 mutants defective in cellulose degradation are deficient in two processive endocellulases, Ce148A and Ce19B, both of which possess a novel modular architecture. J. Bacteriol. 186:136-45.

7. Dodd, D., and I. K. 0. Cann. 2009. Enzymatic deconstruction of xylan for biofuel production GCB Bioenergy 1:2-17.

8. Ezer, A., E. Matalon, S. Jindou, I. Borovok, N. Atamna, Z. Yu, M. Morrison, E. A. Bayer, and R. Lamed. 2008. Cell surface enzyme attachment is mediated by family 37 carbohydrate-binding modules, unique to Ruminococcus albus. J. Bacteriol. 190:8220-2.

9. Flint, H. J., E. A. Bayer, M. T. Rincon, R. Lamed, and B. A. White. 2008. Polysaccharide utilization by gut bacteria: potential for new insights from genomic analysis. Nat. Rev. Microbiol. 6:121-31.

10. Forsberg, C. W., B. Crosby, and D. Y. Thomas. 1986. Potential for manipulation of the rumen fermentation through the use of recombinant DNA techniques. J. Anim. Sci. 63:310-25.

11. Ghosh, D., M. Erman, M. Sawicki, P. Lala, D. R. Weeks, N. Li, W. Pangborn, D. J. Thiel, H. Jornvall, R. Gutierrez, and J. Eyzaguirre. 1999. Determination of a protein structure by iodination: the structure of iodinated acetylxylan esterase. Acta Crystallogr. Sect. D Biol. Crystallogr. 55:779-84.

12. Hakulinen, N., M. Tenkanen, and J. Rouvinen. 2000. Three-dimensional structure of the catalytic core of acetylxylan esterase from Trichoderma reesei: insights into the deacetylation mechanism. J. Struct. Biol. 132:180-90.

13. Henshaw, J., A. Horne-Bitschy, A. L. van Bueren, V. A. Money, D. N. Bolam, M. Czjzek, N. A. Ekborg, R. M. Weiner, S. W. Hutcheson, G. J. Davies, A. B. Boraston, and H. J. Gilbert. 2006. Family 6 carbohydrate binding modules in b-agarases display exquisite selectivity for the non-reducing termini of agarose chains. J. Biol. Chem. 281:17099-107.

14. Henshaw, J. L., D. N. Bolam, V. M. R. Pires, M. Czjzek, B. Henrissat, L. M. A. Ferreira, C. M. G. A. Fontes, and H. J. Gilbert. 2004. The family 6 carbohydrate binding module CmCBM6-2 contains two ligand-binding sites with distinct specificities. J. Biol. Chem. 279:21552-9.

15. Iyo, A. H., and C. W. Forsberg. 1996. Endoglucanase G from Fibrobacter succinogenes S85 belongs to a class of enzymes characterized by a basic C-terminal domain. Can. J. Microbiol. 42:934-43.

16. Kam, D. K., H. S. Jun, J. K. Ha, G. D. Inglis, and C. W. Forsberg. 2005. Characteristics of adjacent family 6 acetylxylan esterases from Fibrobacter succinogenes and the interaction with the XynlOE xylanase in hydrolysis of acetylated xylan. Can. J. Microbiol. 51:821-32.

17. Kelly, S. M., T. J. Jess, and N. C. Price. 2005. How to study proteins by circular dichroism. Biochim. Biophys. Acta 1751:119-39.

18. Koike, S., and Y. Kobayashi. 2001. Development and use of competitive PCR assays for the rumen cellulolytic bacteria: Fibrobacter succinogenes, Ruminococus albus and Ruminococcus flavefaciens. FEMS Microbiol. Lett. 204:361-366.

19. Krause, D. O., S. E. Denman, R. I. Mackie, M. Morrison, A. L. Rae, G. T. Attwood, and C. S. McSweeney. 2003. Opportunities to improve fiber degradation in the rumen: microbiology, ecology, and genomics. FEMS. Microbiol. Rev. 27:663-93.

20. Kumar, R., S. Singh, and O. V. Singh. 2008. Bioconversion of lignocellulosic biomass: biochemical and molecular perspectives. J. Ind. Microbiol Biotechnol 35:377-391.

21. Kyriacou, A., R. J. Neufeld, and C. R. Mackenzie. 1988. Effect of physical parameters on the adsorption characteristics of fractionated Trichoderma reesei cellulase components. Enzyme Microb. Technol. 10:675-681.

22. Lobley, A., L. Whitmore, and B. A. Wallace. 2002. DICHROWEB: an interactive website for the analysis of protein secondary structure from circular dichroism spectra. Bioinformatics 18:211-2.

23. López-Cortes, N., D. Reyes-Duarte, A. Beloqui, J. Polaina, I. Ghazi, O. V. Golyshina, A. Ballesteros, P. N. Golyshin, and M. Ferrer. 2007. Catalytic role of conserved HQGE motif in the CE6 carbohydrate esterase family. FEBS. Lett. 581:4657-62.

24. Lykov, O. P. 1994. Selection of raw material for basic organic synthesis. Chemistry and Technology of Fuels and Oils 30:302-309.

25. Malburg, L. M., Jr., A. H. Iyo, and C. W. Forsberg. 1996. A novel family 9 endoglucanase gene (celD), whose product cleaves substrates mainly to glucose, and its adjacent upstream homolog (celE) from Fibrobacter succinogenes S85. Appl. Environ. Microbiol. 62:898-906.

26. Matte, A., C. W. Forsberg, and A. M. Verrinder Gibbins. 1992. Enzymes associated with metabolism of xylose and other pentoses by Prevotella (Bacteroides) ruminicola strains, Selenomonas ruminantium D, and Fibrobacter succinogenes S85. Can. J. Microbiol. 38:370-6.

27. Miron, J., and D. Ben-Ghedalia. 1993. Digestion of cell-wall monosaccharides of ryegrass and alfalfa hays by the ruminal bacteria Fibrobacter succinogenes and Butyrivibrio fibrisolvens. Can. J. Microbiol. 39:780-6.

28. Pires, V. M. R., J. L. Henshaw, J. A. M. Prates, D. N. Bolam, L. M. A. Ferreira, C. M. G. A. Fontes, B. Henrissat, A. Planas, H. J. Gilbert, and M. Czjzek. 2004. The crystal structure of the family 6 carbohydrate binding module from Cellvibrio mixtus endoglucanase 5A in complex with oligosaccharides reveals two distinct binding sites with different ligand specificities. J. Biol. Chem. 279:21560-8.

29. Qi, M., H. S. Jun, and C. W. Forsberg. 2008. Ce19D, an atypical 1,4-b-D-glucan glucohydrolase from Fibrobacter succinogenes: characteristics, catalytic residues, and synergistic interactions with other cellulases. J. Bacteriol. 190:1976-84.

30. Qi, M., H. S. Jun, and C. W. Forsberg. 2007. Characterization and synergistic interactions of Fibrobacter succinogenes glycoside hydrolases. Appl. Environ. Microbiol. 73:6098-105.

31. Rubin, E. M. 2008. Genomics of cellulosic biofuels. Nature 454:841-5.

32. Scott, H. W., and B. A. Dehority. 1965. Vitamin requirements of several cellulolytic rumen bacteria. J. Bacteriol. 89:1169-75.

33. Somerville, C. 2007. Biofuels. Curr. Biol. 17:R115-9.

34. Somerville, C., S. Bauer, G. Brininstool, M. Facette, T. Hamann, J. Milne, E. Osborne, A. Paredez, S. Persson, T. Raab, S. Vorwerk, and H. Youngs. 2004. Toward a systems approach to understanding plant cell walls. Science 306:2206-11.

35. Stevenson, D. M., and P. J. Weimer. 2007. Dominance of Prevotella and low abundance of classical ruminal bacterial species in the bovine rumen revealed by relative quantification real-time PCR. Appl. Microbiol. Biotechnol. 75:165-174.

36. van Bueren, A. L., C. Morland, H. J. Gilbert, and A. B. Boraston. 2005. Family 6 carbohydrate binding modules recognize the non-reducing end of b-1,3-linked glucans by presenting a unique ligand binding surface. J. Biol. Chem. 280:530-7.

37. Xu, Q., M. Morrison, K. E. Nelson, E. A. Bayer, N. Atamna, and R. Lamed. 2004. A novel family of carbohydrate-binding modules identified with Ruminococcus albus proteins. FEBS Lett. 566:11-6.

TABLE 1
Primers used in this study.

| Primer | Sequence | Experiment |
|---|---|---|
| F1 | 5′-CATATGGCTCCGAACCCGAACTTCCATA | Cloning |
| | TCTACATTGC-3′^{a} | |
| F2 | 5′-CATATGGGCCCGTACACGGACCCGATTG | Cloning |
| | AAATCCCTGGCAAG-3′^{a} | |
| F1′ | 5′-GACGACGACAAGATGGGAATCAAGAATA | Cloning |
| | TCCGC-3′^{b} | |
| R1 | 5′-CTCGAGTTATTCATGTATCACCACCTTT | Cloning |
| | TTTG-3′^{a} | |
| R2 | 5′-CTCGAGCTATCCAATCGGCGGCTGAGCG | Cloning |
| | CTGATTTCCTTGAATTC-3′^{a} | |
| R3 | 5′-CTCGAGCTAGCCATATTCCTCGGGCGGT | Cloning |
| | TCATCCGGAACCGTAG-3′^{a} | |
| R1' | 5′-GAGGAGAAGCCCGGTTATTCATGTATCA | Cloning |
| | CCACCTTTTTTG-3′^{b} | |
| S44G | 5′-CATTGCTTATGGGCAGGGTAACATGGCG | Mutagenesis |
| | GGCAACGGC-3′^{c} | |
| E194N | 5′-CATCTTCCACCAGGGCAACAGTGACGGT | Mutagenesis |
| | ACCGATGC-3′^{c} | |
| E194A | 5′-CATCTTCCACCAGGGCGCAAGTGACGGT | Mutagenesis |
| | ACCGATGC-3′^{c} | |
| D270N | 5′-GCAGGGTAACGGCAAGAATCCGTACCAC | Mutagenesis |
| | TTTGGCCG-3′^{c} | |
| D270A | 5′-GCAGGGTAACGGCAAGGCTCCGTACCAC | Mutagenesis |
| | TTTGGCCG-3′^{c} | |
| H273Q | 5′-CGGCAAGGATCCGTACCAGTTTGGCCGT | Mutagenesis |
| | GCGGGC-3′^{c} | |

^{a}Nucleotides incorporated for restriction enzyme digestion are underlined.
^{b}Nucleotides incorporated for exonuclease digestion are underlined.
^{c}Nucleotides corresponding to the substituted amino acids are underlined.

TABLE 2
Kinetic parameters for FSUAxe6B wild-type
(WT) and its truncational mutants.

| Protein | k_{cat} (s⁻¹)^{a} | Kₘ (mM)^{a} | k_{cat}/Kₘ (s⁻¹ mM⁻¹) |
|---|---|---|---|
| WT | 15 ± 0.3 | 0.08 ± 0.01 | 190 ± 24 |
| TM1 | 15 ± 0.2 | 0.09 ± 0.01 | 170 ± 19 |
| TM2 | 13 ± 0.4 | 0.07 ± 0.01 | 190 ± 27 |

^{a}Data are shown as means ± standard errors.

TABLE 3
Binding parameters of FSUAxe6B wild-type (WT) and its
truncated mutants for insoluble oat-spelt xylan (is-OSX).

| | | qₘₐₓ |
|---|---|---|
| Protein | K_{d} (μM)^{a} | (nmol protein/g is-OSX)^{a} |
| WT | 1.1 ± 0.2 | 100 ± 4 |
| TM3 | 0.83 ± 0.2 | 200 ± 10 |
| TM4 | 1.1 ± 0.2 | 84 ± 3 |
| TM5 | 0.26 ± 0.04 | 350 ± 10 |

^{a}Data are shown as means ± standard errors.

TABLE 4
Kinetic parameters for FSUAxe6B wild-type
(WT) and its site-directed mutants.

| Protein | k_{cat} (s⁻¹)^{a} | Kₘ (mM)^{a} | k_{cat}/Kₘ (s⁻¹ mM⁻¹) |
|---|---|---|---|
| WT | 15 ± 0.3 | 0.08 ± 0.01 | 190 ± 24 |
| S44G | N.D.^{b} | | |
| E194N | 2.8 ± 0.3 | 7 ± 1 | 0.40 ± 0.07 |
| E194A | 2.9 ± 0.1 | 0.2 ± 0.02 | 14 ± 2 |
| D270N | 2.0 ± 0.1 | 0.2 ± 0.03 | 10 ± 2 |
| D270A | 1.8 ± 0.03 | 0.2 ± 0.01 | 9.0 ± 0.5 |
| H273Q | N.D.^{b} | | |
| E194A/D270A | N.D.^{b} | | |

^{a}Data are shown as means ± standard errors.
^{b}N.D., no activity was detected.

TABLE 5
CD spectra for FSUAxe6B wild-type
(WT) and its site-directed mutants.

| | α-helix | β-sheet | β-turn | unordered |
|---|---|---|---|---|
| | (%)^{a} | (%)^{a} | (%)^{a} | (%)^{a} |
| WT | 14 ± 0 | 32 ± 1 | 23 ± 0 | 29 ± 1 |
| S44G | 17 ± 1 | 31 ± 1 | 23 ± 1 | 29 ± 0 |
| E194N | 19 ± 1 | 27 ± 2 | 24 ± 0 | 30 ± 1 |
| E194A | 17 ± 1 | 30 ± 0 | 23 ± 0 | 30 ± 0 |
| D270N | 15 ± 1 | 31 ± 2 | 24 ± 1 | 29 ± 1 |
| D270A | 14 ± 0 | 32 ± 1 | 23 ± 0 | 30 ± 0 |
| H273Q | 13 ± 0 | 34 ± 1 | 23 ± 0 | 30 ± 1 |
| E194A/D270A | 17 ± 0 | 29 ± 0 | 24 ± 0 | 31 ± 1 |

^{a}Data are presented as means ± standard deviations.

1. An isolated polynucleotide comprising a first polynucleotide sequence that encodes SEQ ID NO: 1 wherein said first polynucleotide is linked within one open reading frame to a second polynucleotide sequence to form a linked polynucleotide, wherein SEQ ID NO: 1 binds to a carbohydrate and wherein the linked polynucleotide does not encode a naturally occurring polypeptide.

2. The isolated polynucleotide of claim 1, wherein the first polynucleotide sequence is located within the second polynucleotide sequence.

3. The isolated polynucleotide of claim 1, wherein the first polynucleotide sequence is located at one end of the second polynucleotide sequence.

4. The isolated polynucleotide of claim 1, wherein the first polynucleotide sequence is separated from the second polynucleotide sequence by a polynucleotide encoding a linker.

5. The isolated polynucleotide of claim 1, wherein the isolated polynucleotide comprises multiple copies of the first polynucleotide sequence.

6. The isolated polynucleotide of claim 1, wherein the second polynucleotide sequence encodes a peptide.

7. The isolated polynucleotide of claim 6, wherein the peptide comprises SEQ ID NO: 1.

8. The isolated polynucleotide of claim 1, wherein the second polynucleotide sequence encodes a polypeptide.

9. The isolated polynucleotide of claim 9, wherein the polypeptide comprises an enzyme.

10. The isolated polynucleotide of claim 8, wherein the polypeptide comprises an immunoglobulin or a cytokine.

11. The isolated polynucleotide of claim 1, wherein the second polynucleotide sequence encodes a protein tag.

12. The isolated polynucleotide of claim 11, wherein the protein tag is selected from the group consisting of a Myc tag, a His tag, a maltose binding protein tag, a glutathione-S-transferase tag, an HA tag, a FLAG tag, and a Green fluorescent protein tag.

13. A vector comprising the isolated polynucleotide of claim 1.

14. A host cell comprising the vector of claim 13.

15. A recombinant polypeptide comprising the amino acid sequence encoded by the isolated polynucleotide of claim 1.

16. An isolated polypeptide comprising SEQ ID NO: 1 conjugated to an atom or molecule.

17. The isolated polypeptide of claim 16, wherein the atom or molecule is selected from the group consisting of a fluorophore, a radionuclide, a toxin, a polymer, a fragrance particle, a small molecule, a polypeptide, and a peptide.

18. A method of increasing the ability of a recombinant protein to bind to a carbohydrate, comprising: linking a first isolated polynucleotide encoding SEQ ID NO: 1 to a second isolated polynucleotide encoding a polypeptide, a peptide, or a protein tag to form a linked polynucleotide, wherein the linked polynucleotide encodes a recombinant protein having an increased ability to bind to a carbohydrate compared to the polypeptide, peptide, or protein tag alone.

19. The method of claim 18, further comprising the step of expressing the linked polynucleotides in a host cell, wherein expression of the polynucleotides produces the recombinant protein.

20. A method of increasing the ability of a recombinant protein to bind to a carbohydrate, comprising: linking a first isolated polynucleotide encoding SEQ ID NO: 1 to a second isolated polynucleotide encoding an amino acid sequence selected from a library of amino acid sequences to form a linked polynucleotide, wherein the linked polynucleotide encodes a recombinant protein having an increased ability to bind to a carbohydrate compared to the amino acid sequence alone.

21. A method of identifying a protein having an ability to bind a carbohydrate, comprising
providing a labeled polynucleotide, wherein the polynucleotide encodes SEQ ID NO: 1;
hybridizing the labeled polynucleotide to a homologous sequence in a nucleotide library; and
isolating the sequence bound by the labeled polynucleotide, wherein the sequence encodes a protein having an ability to bind to a carbohydrate.

22. The method of claim 21, wherein the nucleotide library is a cDNA library, or a genomic library.
